(19) United States
(12) Patent Application Publication
(10) Pub. No.: US 2023/0064978 A1
(43) Pub. Date: Mar. 2, 2023

(54) CYCLIC PEPTIDE RECEPTOR LANTHIONINE SYNTHETASE C-LIKE PROTEIN (LANCL) AND USES THEREOF

(71) Applicant: LATERAL IP PTY LTD, Melbourne, Victoria (AU)

(72) Inventors: Andrew Gearing, Melbourne, Victoria (AU); David KENLEY, Melbourne, Victoria (AU)

(73) Assignee: LATERAL IP PTY LTD, Melbourne, Victoria (AU)

(21) Appl. No.: 17/788,845

(22) PCT Filed: Dec. 29, 2020
(86) PCT No.: PCT/AU2020/051442

§ 371 Date: Jun. 24, 2022

(30) Foreign Application Priority Data
Dec. 27, 2019 (AU) 2019904956

(51) Int. Cl. A61K 38/12; A61K 31/485; A61K 31/616; A61P 25/04; A61P 29/02

(52) U.S. Cl. CPC A61K 38/12; A61K 31/485; A61K 31/616; A61P 25/04; A61P 29/02

(57) ABSTRACT

The present disclosure relates generally to a method of screening for and identifying a ligand of a Lanthionine synthetase C-like protein (LANCL) and the use of the identified ligands for the treatment of conditions, including pain. Also disclosed herein is a method of treating a condition, including pain, comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising SEQ ID NO:1 (YLRIVQCRSVEGSCGF).

### FIELD OF THE INVENTION

The invention relates generally to a cyclic peptide receptor and uses thereof for screening and identifying therapeutic agents, and to the use of the identified therapeutic agents, including for the treatment of pain.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Pain can be a debilitating sensory experience that is typically associated with tissue damage and/or an underlying neurological disorder. Pain, whether acute or chronic, may arise in the absence of any detectable stimulus, injury or underlying disease. Acute pain usually lasts for short periods (e.g., a few hours or days), and will typically disappear upon cessation of the underlying stimulus. By contrast, chronic pain lasts for longer periods (e.g., weeks or months), and can persist even in the absence of an underlying stimulus.

There are two widely recognized types of pain—nociceptive pain and neuropathic pain. Nociceptive pain is the result of potentially harmful stimulation of the sensory nerve fibres, detected by nociceptors around the body that respond to mechanical or physical damage. Nociceptive pain serves a protective biological function by warning of tissue damage, to cause withdrawal from the noxious stimulus. Nociceptive pain may result from thermal damage, such as burns or frostbite, or result from mechanical trauma such as laceration or pressure.

In contrast to nociceptive pain, neuropathic pain is caused by a primary lesion, malfunction or dysfunction in the peripheral or central nervous system. Neuropathic pain has no protective effect and can develop days or months after an injury or after resolution of a disease state, and is frequently long-lasting and chronic.

Neuropathic pain may result from nerve damage caused by a trauma such as a sporting injury, an accident, a fall or a penetrating injury or the nerve damage may result from a disease process such as stroke, viral infections, exposure to toxins, degenerative diseases and diabetes.

In some instances, nociceptive pain and neuropathic pain can overlap, whereby some injuries or disease states can affect both tissues and nerves, giving rise to both types of pain.

Although there are many available analgesic remedies, the choice of analgesia and its effectiveness is typically dependent on the level of reported pain, whether acute or chronic, the location and the type of pain, and/or potential contraindications. According to the World Health Organisation (WHO) analgesic ladder, mild or transient pain conditions should respond to over-the-counter medication, such as paracetamol and non-steroidal anti-inflammatory drugs such as aspirin, ibuprofen and naproxen. Treatment of moderate to severe, chronic or complex pain, is currently reliant on the use of opioid-based analgesics, including mild codeine, oxycodone, methadone, morphine and fentanyl.

Non-opioid analgesics, such as selective COX-2 inhibitors have been withdrawn due to unacceptable associated risks to cardiovascular disease. The use of medicinal cannabis and cannabinoids remain controversial with little evidence supporting its broad use for treating pain, in particular chronic pain. Therefore, opioid analgesics remain the gold standard. However, opioid analgesics carry significant side effects such as sedation and sleepiness and in the case of long term use, the risk of drug tolerance and drug dependence. Accordingly, there remains an urgent need for new and alternative options that are effective for treating or preventing pain, whilst limiting some of the adverse side effects seen with many other analgesics. To this end, the present inventors have previously identified a class of cyclic peptides capable of treating pain, including neuropathic pain. The present inventors have now, for the first time, identified and characterised the molecular target of these cyclic peptides, allowing for the identification and use of a new class of therapeutic agents for treating pain, including neuropathic pain. The inventor's findings also allow for the identification and use of a new class of therapeutic agents for treating conditions other than pain, including conditions for which treatment has previously been ascribed to the peptides described herein.

### SUMMARY OF THE INVENTION

The present invention is predicated, at least in part, on the inventors' identification and characterisation of the molecular target (receptor) of a new class of cyclic peptide molecules to which analgesic properties have previously been ascribed. This is the first time that the molecular target of this new class of cyclic peptide molecules has been identified and characterised, allowing for new and improved therapeutic agents to be identified, including therapeutic agents capable of treating conditions such as pain.

Accordingly, in one aspect, the present invention provides a method of treating pain in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising SEQ ID NO:1 (YLRIVQCRSVEGSCGF).

Also disclosed herein is a method of screening for analgesic agents, the method comprising: (a) contacting a candidate agent with a Lanthionine synthetase C-like protein (LANCL) in the presence of a peptide comprising SEQ ID NO:1 (YLRIVQCRSVEGSCGF), or a structural analogue thereof, and under conditions that would allow binding of the candidate agent to the LANCL, and (b) determining whether the candidate agent binds to the LANCL and competes for binding to the LANCL with the peptide comprising SEQ ID NO:1 or with the structural analogue thereof, wherein the ability of the candidate agent to compete for binding to the LANCL with the peptide comprising SEQ ID NO:1, or with the structural analogue thereof, is indicative that the candidate agent is an analgesic agent.

Also disclosed herein is a method of screening for a ligand of a Lanthionine synthetase C-like protein (LANCL), the method comprising: (a) contacting a candidate agent with the LANCL in the presence of a cyclic peptide comprising SEQ ID NO:1, or a structural analogue thereof, and under conditions that would allow binding of the candidate agent to the LANCL, and (b) determining whether the candidate agent binds to the LANCL and competes for binding to the LANCL with the cyclic peptide comprising SEQ ID NO: 1 or with the structural analogue thereof, wherein the ability of the candidate agent to compete for binding to the LANCL with the cyclic peptide comprising SEQ ID NO:1, or with the structural analogue thereof, is indicative that the candidate agent is a ligand of LANCL.

In an embodiment, the LANCL is selected from the group consisting of LANCL1, LANCL2 and LANCL3. In a preferred embodiment, the LANCL is LANCL1.

In another aspect, there is provided a composition comprising the analgesic agent identified by the screening methods disclosed herein, wherein the agent is not a peptide derived from human growth hormone or from a non-human homologue thereof.

Also disclosed herein is a composition for use in treating pain in a subject in need thereof, the composition comprising an agent that binds to Lanthionine synthetase C-like protein 1 (LANCL1), and competes for binding to LANCL1 with a cyclic peptide comprising SEQ ID NO:1, or with a structural analogue thereof, and wherein the agent is not a peptide derived from human growth hormone or from a non-human homologue thereof.

The present disclosure also extends to use of an agent that binds to Lanthionine synthetase C-like protein 1 (LANCL1) and competes for binding to LANCL1 with a cyclic peptide of SEQ ID NO:1, or with a structural analogue thereof, in the manufacture of a medicament for treating pain in a subject in need thereof, wherein the agent is not a peptide derived from human growth hormone or from a non-human homologue thereof.

The present disclosure also extends to a method of treating a condition in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising SEQ ID NO:1 (YLRIVQCRSVEGSCGF), wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

Also disclosed herein is a composition for use in treating a condition in a subject in need thereof, the composition comprising an agent that binds to Lanthionine synthetase C-like protein 1 (LANCL1), and competes for binding to LANCL1 with a cyclic peptide comprising SEQ ID NO:1, or with a structural analogue thereof, and wherein the agent is not a peptide derived from human growth hormone or from a non-human homologue thereof, wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

The present disclosure also extends to use of an agent that binds to Lanthionine synthetase C-like protein 1 (LANCL1) and competes for binding to LANCL1 with a cyclic peptide of SEQ ID NO:1, or with a structural analogue thereof, in the manufacture of a medicament for treating a condition in a subject in need thereof, wherein the agent is not a peptide derived from human growth hormone or from a non-human homologue thereof and wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

### BRIEF DESCRIPTION OF THE DRAWINGS

FIG. 1 shows binding of the LAT9991-PAL ligand (SEQ ID NO:12-PAL; 5 μM) to Dorsal root ganglion (DRG) neurons from mice either treated (top) or untreated (bottom) with paclitaxel—the red staining reveals selective binding of LAT9991-PAL only to neurons from treated and neuropathic animals. Scale bar represents 10 μm.

FIG. 2 is a high-powered image of a photomicrograph showing that the molecular target of the LAT9991-PAL (red staining in images) in neurons from neuropathic mice following nerve constriction is expressed just inside the neuronal cell membrane and as punctate staining in the cytoplasm. Scale bar represents 10 μm.

FIG. 3 shows the gel separation of complexes formed by cross-linked LAT9991-PAL (SEQ ID NO: 12-PAL; 5 μM) in tissue homogenates of nerve tissue from neuropathic animals. The results reveal specific patterns of staining that identify targets at three molecular weight ranges: 12-15 kDa, 37 kDa and 40-50 kDa. Homogenates in the absence of PAL ligand and homogenates with LAT9991-PAL (5 μM) in the presence of an excess of LAT8881 (SEQ ID NO:1; 50 μM) were used as controls.

FIG. 4 shows that LAT9991-PAL, LAT9993-PAL (SEQ ID NO:41-PAL) and LAT7771-PAL (SEQ ID NO:52-PAL) bound to LANCL1 (identified by mass spectroscopy) from tissue homogenates and that this binding was inhibited in the presence of excess LAT8881.

FIG. 5 shows detection of LANCL1 using a commercial LANCL1 antibody in Western Blot analysis to confirm the identity of the putative target protein binding to LAT9991-PAL.

FIG. 6 shows that LAT9991-PAL bound to recombinant LANCL1 and that this binding was inhibited in the presence of excess LAT8881.

FIG. 7 shows that LAT7771-PAL bound to recombinant LANCL1 and that this binding was inhibited in the presence of excess LAT8881.

FIG. 8 shows that LAT9993S-PAL (SEQ ID NO:44-PAL) bound to recombinant LANCL1 and that this binding was inhibited in the presence of excess LAT8881.

FIG. 9 shows that LAT9991-PAL bound to recombinant LANCL1 and that this binding was inhibited in the presence of (from left to right) excess LAT8881, LAT9991, LAT7771 and LAT9993S.

FIG. 10 shows that LAT7771-PAL bound to recombinant LANCL2 and that this binding was inhibited in the presence of excess LAT8881.

FIG. 11 shows that LAT9993-PAL bound to recombinant LANCL3 and that this binding was inhibited in the presence of excess LAT8881.

FIG. 12 shows the binding site of LANCL1 for the cyclic peptide LAT9991-PAL following dependent peptide MS analysis of the modified peptide, IDPHAPNEM(Ox)LYGR (SEQ ID NO:59). The modified peptide was identified with a characteristic ΔM of 454.199 (PAL-CR-N₂).

FIG. 13 shows loss of LANCL1 in adenocarcinomic alveolar basal epithelial cells (A549) following gene silencing by siRNA (SiLANCL1) as compared to A549 cells transfected with a control siRNA (SiControl). After 48 hours of LANCL1 silencing, LANCL1 protein was depleted, as evidenced by a loss of the ˜37 kDa LANCL1 protein from cell isolates as determined by Western blot analysis using an anti-LANCL1 antibody (Invitrogen) (A). This was consistent with the observed decrease in LANCL1 staining as determined by confocal microscopy (B) using an anti-LANCL1 antibody (Invitrogen), although some endogenous staining of LANCL1 in the cytosol of SiLANCL A549 cells remained.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The singular terms “a”, “an” and “the” include plural referents unless context clearly indicates otherwise. It is further to be understood that all base sizes or amino acid sizes, and all molecular weight or molecular mass values, given for nucleic acids or polypeptides are approximate, and are provided for description.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “and/or” refers to and encompasses any and all possible combinations of one or more of the associated listed items, as well as the lack of combinations when interpreted in the alternative (or).

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Each embodiment described herein is to be applied mutatis mutandis to each and every embodiment unless specifically stated otherwise.

### Screening Methods

As noted elsewhere herein, the inventor has, for the first time, identified and characterised the molecular target of a class of cyclic peptide molecules to which analgesic properties have previously been ascribed, allowing for methods for screening candidate compounds that can be used to treat pain, in particular neuropathic pain. Thus, disclosed herein is a method of screening for analgesic agents, the method comprising: (a) contacting a candidate agent with a Lanthionine synthetase C-like protein (LANCL) in the presence of a peptide comprising SEQ ID NO:1 (YLRIVQCRSVEGSCGF), or a structural analogue thereof, and under conditions that would allow binding of the candidate agent to the LANCL, and (b) determining whether the candidate agent binds to the LANCL and competes for binding to the LANCL with the peptide comprising SEQ ID NO:1 or with the structural analogue thereof, wherein the ability of the candidate agent to compete for binding to the LANCL with the peptide comprising SEQ ID NO:1, or with the structural analogue thereof, is indicative that the candidate agent is an analgesic agent. In an embodiment, the LANCL is selected from the group consisting of LANCL1, LANCL2 and LANCL3. In a preferred embodiment, the LANCL is LANCL1.

The inventor's findings also allow for general methods for screening for ligands of LANCL. Thus, also disclosed herein is a method of screening for a ligand of LANCL, the method comprising: (a) contacting a candidate agent with the LANCL in the presence of a cyclic peptide comprising SEQ ID NO:1, or a structural analogue thereof, and under conditions that would allow binding of the candidate agent to the LANCL, and (b) determining whether the candidate agent binds to the LANCL and competes for binding to the LANCL with the cyclic peptide comprising SEQ ID NO:1 or with the structural analogue thereof, wherein the ability of the candidate agent to compete for binding to the LANCL with the cyclic peptide comprising SEQ ID NO:1, or with the structural analogue thereof, is indicative that the candidate agent is a ligand of LANCL In some embodiments, the identified ligand is a therapeutic agent that may be employed to treat conditions that may benefit from the modulation of LANCL activity, illustrative examples of which include sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, pain (e.g., neuropathic pain) and a bone disorder.

Lanthionine synthetase C-like proteins (LANCL) are eukaryotic homologs of bacterial LanC, a cyclase involved in the formation of lanthionine rings in lantibiotics. Humans have three LanCL proteins—LANCL1, LANCL2, and LANCL3, encoded on chromosomes 2, 7, and X, respectively. The first member of the eukaryotic LANC-like (LANCL) protein family, LANCL1, was isolated from human erythrocyte membranes. LANCL2, was subsequently identified in humans. LANCL1 homologs have also been found and characterized in other mammals, including mouse, rat and cattle. A comparison of the amino acid sequences of eukaryotic LANCL proteins with prokaryotic LANC enzymes revealed several critical similarities, and these have formed the foundation for the definition of LANCL proteins in eukaryotes. For instance, LANCL proteins contain seven hydrophobic sequence repeats, each with one characteristic GXXG consensus motif (where G=glycine and X=any amino acid). Further, LANCL proteins contain three critical motifs, a histidine (H)-glycine (G) motif in repeat 4, a tryptophan (W)-cysteine (C)-X-glycine (G) motif in repeat 5, and a cysteine (C)-histidine (H)-glycine (G) motif in repeat 6. As revealed by the crystal structure of the Lactococcus lactis LanC protein, nisin cyclise (NisC), and by site-directed mutagenesis studies, the HG motif of repeat 4 is critical for substrate deprotonation in order to allow correct cyclization, and the WCXG motif of repeat 5 and CHG motif of repeat 6 contain conserved zinc-coordinating resides (see Chen and Ellis, Plant Signal Behav. 2008; 3(5): 307-310).

Lanthionine synthetase C-like protein 1 (LANCL1) is a 399 amino acid peripheral membrane protein that is understood to be one of three related mammalian LANCL proteins (LANCL1, 2 and 3), originally discovered as a membrane-associated erythrocyte GPCR p40/GPR69A with sequence and structural homology to a bacterial enzyme lanthionine synthetase component C (LANC), which is involved in antibiotic synthesis. LANC is a zinc-containing enzyme that acts in concert with specific dehydratases to facilitate intramolecular conjugation of cysteine to serine or threonine residues, yielding macrocyclic thioether analogs of cysteine known as lanthionines. These products display potent antimicrobial activity and are also known as lantibiotics. Subsequent studies have shown that LANCL1 in animals is not a GPCR and does not possess the cyclase activities of the bacterial enzyme.

The genomic region of human chromosome 2q34 proximal to LANCL1 has been implicated in early-onset AD susceptibility forms of familial schizophrenia and neural tube defects. LANCL1 has also been implicated in a genetic association study with asthma. LANCL1 has also been identified as one of three genes in mice that correlated with susceptibility to MTMP neurotoxicity toxicity (a model for Parkinson disease).

LANCL2 has been shown to be involved in the abscisic acid (ABA) signaling pathway, acting downstream to propagate ABA-specific effects in immune and insulinoma cells. Previous studies have reported that LANCL2 regulates Akt, specifically involved in facilitating optimal phosphorylation of Akt by mammalian target of rapamycin complex 2 (mTORC2), through direct physical interaction with both the kinase and the substrate. Human LANCL2 has also been suggested to have a role in adriamycin sensitizing. LANCL2 is highly expressed in the testis and the brain, with lesser, albeit ubiquitous expression in all other tissues examined. Comparatively little is known about LANCL3.

As used herein, the term “LANCL1” includes functional variants thereof that differ structurally (e.g., by amino acid sequence) from a native LANCL1 polypeptide but retain, or substantially retain the biological activity of the native protein. In an embodiment, LANCL1 is a human LANCL1. Examples of human LANCL1 molecules will be known to persons skilled in the art, illustrative examples of which are described in GenBank Accession number CAG46576 (version CAG46576.1) and GenBank Accession no. NP_006046.1. Thus, in an embodiment, the LANCL1 comprises the amino acid sequence of GenBank Accession number CAG46576 (version CAG46576.1) or GenBank Accession no. NP_006046.1, or an amino acid sequence having at least 70% sequence identity or similarity to any of the foregoing.

The term “LANCL1” also encompasses non-human homologues, such as murine, canine, feline and equine LANCL1 isoforms. Non-human isoforms of LANCL1 will be known to persons skilled in the art, illustrative examples of which are described in GenBank Accession numbers RLQ75574.1, OWK13010.1, NP_001177913.1, NP_001177914.1 and NP_067270.1.

As used herein, the tem “LANCL2” includes functional variants thereof that differ structurally (e.g., by amino acid sequence) from a native LANCL2 polypeptide but retain, or substantially retain the biological activity of the native protein. In an embodiment, LANCL2 is a human LANCL2. Examples of human LANCL2 molecules will be known to persons skilled in the art, an illustrative example of which is described in GenBank Accession number NP_061167.1 (version NP_061167.1). Thus, in an embodiment, the LANCL2 comprises the amino acid sequence of GenBank Accession number NP_061167.1 (version NP_061167.1), or an amino acid sequence having at least 70% sequence identity or similarity to any of the foregoing.

The term “LANCL2” also encompasses non-human homologues, such as murine, canine, feline and equine LANCL2 isoforms. Non-human isoforms of LANCL2 will be known to persons skilled in the art, illustrative examples of which are described in GenBank Accession numbers AAH16072.1, AAI49312.1, and NP_001014209.1.

As used herein, the term “LANCL3” includes functional variants thereof that differ structurally (e.g., by amino acid sequence) from a native LANCL3 polypeptide but retain, or substantially retain the biological activity of the native protein. In an embodiment, LANCL3 is a human LANCL3. Examples of human LANCL3 molecules will be known to persons skilled in the art, an illustrative example of which is described in GenBank Accession number NP_001163802 (version NP_001163802.1). Thus, in an embodiment, the LANCL3 comprises the amino acid sequence of GenBank Accession number NP_001163802 (version NP_001163802.1), or an amino acid sequence having at least 70% sequence identity or similarity to any of the foregoing.

The term “LANCL3” also encompasses non-human homologues, such as murine, canine, feline and equine LANCL3 isoforms. Non-human isoforms of LANCL3 will be known to persons skilled in the art, illustrative examples of which are described in GenBank Accession numbers NP_775590.2, XP_031301363.1 and XP_018875202.2.

The term “functional variant” is used herein to denote molecules that differ from the native LANCL1 peptide (of human or non-human isoforms) by one or more amino acid insertions, deletions and/or substitutions, either conservative or non-conservative, wherein the variant retains, or substantially retains, the biological activity of the native protein, including its ability to bind to a cyclic peptide comprising SEQ ID NO:1, or to a structural analogue thereof, as described elsewhere herein. Methods for determining whether a peptide is a functional variant of LANCL1, LANCL2 and/or LANCL3 will be familiar to persons skilled in the art, an illustrative example of which is described elsewhere herein (e.g., the ability of the functional variant to bind to and/or form a complex with a cyclic peptide comprising SEQ ID NO:1, or to a structural analogue thereof, as described elsewhere herein). Functional variants of LANCL1, LANCL2 and/or LANCL3 also extend to functional fragments of LANCL1, LANCL2 and/or LANCL3, respectively. It is to be understood that a functional fragment can be any suitable length, as long as it retains, or substantially retains, the biological activity of the native protein, including its ability to bind to a cyclic peptide comprising SEQ ID NO:1, or to a structural analogue thereof, as described elsewhere herein. In an embodiment, the functional fragment is at least 50 amino acid residues in length (i.e., 50, 51, 52, 53, 54, 55, 56, 57, 58 and so on), preferably at least 100 amino acid residues in length, preferably at least 150 amino acid residues in length, preferably at least 200 amino acid residues in length, preferably at least 250 amino acid residues in length, preferably at least 300 amino acid residues in length, preferably at least 350 amino acid residues in length, or more preferably at least 350 amino acid residues in length.

The terms “native”, “wild-type”, “naturally occurring” and the like are used interchangeably herein to refer to a gene or gene product that has the characteristics of that gene or gene product when isolated from a naturally occurring source. A wild type, native or naturally occurring gene or gene product (e.g., a polypeptide) is that which is most frequently observed in a population and is thus arbitrarily designated the “normal” or “wild-type” form of the gene or gene product.

As noted elsewhere herein, functional variants of LANCL1, LANCL2 and LANCL3 encompass, without limitation, molecules having an amino acid sequence that shares at least 70% (and at least 71% to at least 99% and all integer percentages in between) sequence identity or similarity with a native LANCL sequence. Illustrative examples of native LANCL polypeptide sequences are described elsewhere herein, including, but not limited to, GenBank Accession numbers CAG46576 (e.g., version CAG46576.1), NP_006046.1, RLQ75574.1, OWK13010.1, NP_001177913.1, NP_001177914.1 and NP_067270.1. Functional variants of LANCL1, LANCL2 and LANCL3 further encompass natural allelic variation of the LANCL peptides that may exist and occur from one organism to another. The degree and location of glycosylation or other post-translation modifications may also vary depending on the chosen host and the nature of the hosts cellular environment. Functional variants of LANCL1, LANCL2 and LANCL3 also extend to LANCL peptides that have either been chemically modified (e.g. phosphorylation, methylation or acetylation) relative to a reference or naturally-occurring (native) LANCL peptide and/or contain one or more amino acid sequence alterations relative to a reference or naturally-occurring LANCL peptide and/or contain truncated amino acid sequences relative to a reference or naturally-occurring full-length or processed LANCL peptide, as described elsewhere herein. Functional variants of LANCL1, LANCL2 and LANCL3 also encompass proteinaceous molecules with a slightly modified amino acid sequence, for instance, peptides having a modified N-terminal end including N-terminal amino acid deletions or additions, and/or polypeptides that have been chemically modified relative to a reference or naturally-occurring LANCL peptide. As noted elsewhere herein, functional variants of LANCL also extend to peptides having an amino acid sequence that differs from the sequence of a reference or naturally-occurring LANCL peptide by insertion, deletion, or substitution of one or more amino acids, as described elsewhere herein.

A “conservative amino acid substitution” is to be understood as meaning a substitution in which the amino acid residue is replaced with an amino acid residue having a similar side chain. Families of amino acid residues having similar side chains have been defined in the art, which can be generally sub-classified as shown in Table 1, below:

TABLE 1
AMINO ACID SUB-CLASSIFICATION

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, Threonine |
| Polar/large | Asparagine, Glutamine |
| Hydrophobic | Tyrosine, Valine, Isoleucine, Leucine, Methionine, Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Conservative substitutions are shown in Table 2, below, under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

There are numerous approaches to screening candidate agents for their ability to (i) bind to LANCL1, LANCL2 and/or LANCL3 and (ii) compete for binding with a cyclic peptide of SEQ ID NO:1 or a structural analogue thereof. A variety of screening assays or methods will suffice and, in light of the present disclosure, those not expressly described herein will nevertheless be comprehended by one of ordinary skill in the art.

TABLE 2
EXEMPLARY AND PREFERRED
AMINO ACID SUBSTITUTIONS

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

As described herein, the candidate compounds (agents) may be created by any combinatorial chemical method. Alternatively, the candidate agents may be naturally occurring molecules that are extracted and purified from a suitable source, or synthesized in vivo or in vitro. Candidate agents (compounds) to be tested (screened) can be produced, for example, by bacteria, yeast, plants or other organisms (e.g., natural products), produced chemically (e.g., small molecules, including peptidomimetics), or produced recombinantly. Candidate compounds include non-peptidyl organic molecules, peptides, polypeptides, peptidomimetics, sugars and hormones.

The term “agent”, as used herein, includes a compound that induces a desired pharmacological and/or physiological effect. The term also encompass pharmaceutically acceptable and pharmacologically active ingredients of those compounds specifically mentioned herein including but not limited to, salts, esters, amides, prodrugs, active metabolites, analogs and the like. When the above term is used, it is to be understood that it includes the active agent per se, as well as pharmaceutically acceptable, pharmacologically active salts, esters, amides, prodrugs, metabolites, analogs, etc. The term “agent” is not to be construed narrowly, but extends to small molecules, proteinaceous molecules such as peptides, polypeptides and proteins as well as compositions comprising them, and chemical analogs thereof, as well as cellular agents. The term “agent” includes a cell that is capable of producing and secreting the agent referred to herein, as well as a polynucleotide comprising a nucleotide sequence that encodes that agent. Thus, the term “agent” extends to nucleic acid constructs including vectors such as viral or non-viral vectors, expression vectors and plasmids for expression in and secretion in a range of cells. The terms “candidate agent” and “test agent” are used interchangeably herein to refer to agents and/or compositions that are to be screened for their ability to bind to Lanthionine synthetase C-like protein 1 (LANCL1), as herein described.

The candidate compounds can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of candidate compounds to the screening methods disclosed herein can be in either an isolated form or as mixtures of compounds, especially in initial screening steps. Optionally, the candidate compounds may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug screening programs that test libraries of compounds and natural extracts, high throughput assays are desirable in order to maximize the number of compounds surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target which is mediated by a candidate compound. Moreover, the effects of cellular toxicity or bioavailability of the candidate compound can be generally ignored in the in vitro system, the assay instead being focused primarily on the effect of a candidate compound on the molecular target (i.e., LANCL1).

Candidate compounds may also be selected by electronic screening of well-known large compound libraries, such as the Available Chemical Directory (ACD; http://www.organicworldwide.net/content/available-chemical-directory). Compounds of such libraries may be analyzed by docking programs. In particular, to evaluate the quality of fit and strength of interactions between a candidate compound and LANCL1, docking programs such as Autodock (available from Oxford Molecular, Oxford, UK), Dock (available from Molecular Design Institute, University of California San Francisco, Calif.), Gold (available from Cambridge Crystallographic Data Centre, Cambridge, UK) and FlexX and FlexiDock (both available from Tripos, St. Louis, Mo.) may be used. These programs and the program Affinity (available from Molecular Simulations, San Diego, Calif.) may also be used in further development and optimization of candidate compounds. Standard molecular mechanics force fields such as CHARMm and AMBER may also be used in energy minimization and molecular dynamics.

In an illustrative example of a screening assay of the present invention, the candidate compound is contacted with a LANCL1 peptide. In an embodiment, the LANCL1 comprises the amino acid sequence: MAQRAFPNPY ADYNKSLAEG YFDAAGRLTP EFSQRLTNKI RELLQQMERG PKSADPRDGT GYTGWAGIAV LYLHLYDVFG DPAYLQLAHG YVKQSLNCLT KRSITFLCGD AGPLAVAAVL YHKMNNEKQA EDCITRLIHL NKIDPHAPNE MLYGRIGYIY ALLFVNKNFG VEKIPQSHIQ QICETILTSG ENLARKRNFT AKSPLMYEWY QEYYVGAAHG LAGIYYYLMQ PSLQVSQGKL HSLVKPSVDY VCQLKFPSGN YPPCIGDNRD LLVHWCHGAP GVIYMLIQAY KVFREEKYLC DAYQCADVIW QYGLLKKGYG LCHGSAGNAY AFLTLYNLTQ DMKYLYRACK FAEWCLEYGE HGCRTPDTPF SLFEGMAGTI YFLADLLVPT KARFPAFEL (SEQ ID NO:2), as described in GenBank Accession No. CAG46576 (version CAG46576.1).

In another illustrative example of a screening assay of the present invention, the candidate compound is contacted with a LANCL2 peptide. In an embodiment, the LANCL2 comprises the amino acid sequence of GenBank Accession No. NP_061167 (version NP_061167.1).

In another illustrative example of a screening assay of the present invention, the candidate compound is contacted with a LANCL3 peptide. In an embodiment, the LANCL3 comprises the amino acid sequence of GenBank Accession No. NP_001163802 (version NP_001163802.1).

In another illustrative example of a screening assay of the present invention, the candidate compound is contacted with two or more of LANCL1, LANCL2 and LANCL3 peptides (g, (i) LANCL1 and LANCL2; (ii) LANCL1, LANCL2 and LANCL3; (iii) LANCL1 and LANCL3; (iv) LANCL2 and LANCL3).

The LANCL1, LANCL2 and LANCL3 may be conveniently prepared by recombinant techniques using standard protocols as described, for example, in Ausubel et al. “Current Protocols in Molecular Biology”, John Wiley & Sons Inc, 1994-2003. For example, the LANCL1, LANCL2 and LANCL3 may be prepared by a procedure including the steps of: (a) preparing a construct comprising a coding sequence for LANCL1, LANCL2 and/or LANCL3, wherein the coding sequence is operably connected to a regulatory element; (b) introducing the construct into a host cell; (c) culturing the host cell to express the coding sequence to thereby produce the encoded peptide; and (d) isolating the encoded LANCL peptide from the host cell. The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

A representative LANCL1 coding sequence comprises the nucleotide sequence of GenBank Accession No. NM_006055 (version NM_006055.3), or a sequence corresponding thereto, including codon-optimized sequences for enhanced expression in a selected host cell. A representative LANCL2 coding sequence comprises the nucleotide sequence of GenBank Accession No. NM_018697.3, or a sequence corresponding thereto, including codon-optimized sequences for enhanced expression in a selected host cell. A representative LANCL3 coding sequence comprises the nucleotide sequence of GenBank Accession No. NM_001170331.2, or a sequence corresponding thereto, including codon-optimized sequences for enhanced expression in a selected host cell.

In an embodiment, to the mixture of the candidate compound and LANCL is added a composition containing a cyclic peptide comprising SEQ ID NO:1, or a structural analogue thereof. Detection and quantification of complexes formed between LANCL1 and the cyclic peptide, or structural homologue thereof, provides a means for determining the candidate compound's ability to inhibit the formation of the complex between LANCL and the cyclic peptide, or structural analogue thereof. The efficacy of the candidate compound to inhibit the formation of a complex between LANCL and the cyclic peptide, or structural analogue thereof can be assessed by generating dose response curves from data obtained using various concentrations of the candidate compound. A control assay can also be performed to provide a baseline for comparison. For example, in a control assay, LANCL1 is added to a composition comprising the cyclic peptide or structural analogue thereof, and the formation of a LANCL1/cyclic peptide complex is quantitated in the absence of the candidate compound. It will be understood that, in general, the order in which the reactants may be admixed can be varied, and can be admixed simultaneously. Moreover, in place of purified proteins, cellular extracts and lysates may be used to render a suitable cell-free assay system.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favoured (e.g., compared to a non-aggregated, or non-complexed, state of its component molecules). As used herein the term “complex”, unless described as otherwise, refers to the assemblage of two or more molecules (e.g., peptides, polypeptides or a combination thereof). The term “interaction”, including its grammatical equivalents, when referring to an interaction between two molecules, refers to the physical contact of the molecules with one another. Generally, such an interaction results in an activity (which produces a biological effect) of one or both of said molecules. The physical contact typically requires binding or association of the molecules with one another and may involve the formation of an induced magnetic field or paramagnetic field, covalent bond formation, ionic interaction (such as, for example, as occurs in an ionic lattice), a hydrogen bond, or alternatively, a van der Waals interaction such as, for example, a dipole-dipole interaction, dipole-induced dipole interaction, induced dipole-induced dipole interaction, or a repulsive interaction, or any combination of the above forces of attraction.

The capacity of a candidate compound to modulate (compete with) the interaction between LANCL and the cyclic peptide, or a structural analogue thereof, may be tested by any method known to the person of skill in the art to be suitable for assessing the interaction between two proteins. Illustrative examples include immunoblotting, immunoprecipitation analyses, immunofluorescence of PAR foci, fluorescence polarization, FRET (Fluorescence Resonance Energy Transfer), BRET (Bioluminescence Resonance Energy Transfer), AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIAcore™) isothermal titration calorimetry (ITC), differential scanning calorimetry, microscale thermophoresis, gel electrophoresis, and chromatography including gel filtration. These and other methods, including those described elsewhere herein, may take advantage of a fusion partner or label of LANCL1, LANCL2 and/or LANCL3 and/or the cyclic peptide or structural analogue thereof (e.g., photo-activated labelling (PAL), as described elsewhere herein). Assays may employ a variety of detection methods including, but not limited to, chromogenic, fluorescent, luminescent, or isotopic labels.

As used herein, the term “modulate” means to cause or facilitate a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon.

The present disclosure also contemplates the use of an interaction trap assay, also known as the “two hybrid assay”, for identifying candidate compounds that disrupt the interaction between LANCL and a cyclic peptide comprising SEQ ID NO:1, or a structural analogue thereof (see, for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In an embodiment, the present disclosure contemplates the use of reverse two hybrid systems to identify compounds (e.g., small molecules or peptides) that dissociate interactions between LANCL and a cyclic peptide comprising SEQ ID NO:1 or a structural analogue thereof (see, for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368).

Candidate compounds may be further tested in animal models to identify those compounds having the desired therapeutic profile in vivo. Those candidates may serve as “lead compounds” for the further development of pharmaceuticals by, for example, subjecting the compounds to sequential modifications, molecular modelling, and other routine procedures employed in rational drug design.

It is to be understood that the term “structural analogue”, insofar as it refers to cyclic peptides comprising SEQ ID NO:1, means molecules that differ from the amino acid sequence of SEQ ID NO:1 by one or more amino acid insertions, deletions and/or substitutions, either conservative or non-conservative, but would otherwise retain, or substantially retain, a cyclic confirmation (e.g., by retaining the disulphide bond between cysteine residues corresponding to the two cysteine residues of SEQ ID NO:1; YLRIVQCRSVEGSCGF) and the ability to bind to LANCL1, LANCL2 or LANCL3, as described herein. In this context, and in the absence of any explicit indication to the contrary, the terms “structural analogue” and “functional analogue” are used interchangeably herein. By “corresponds to” or “corresponding to” is meant an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence. In an embodiment, the structural analogue comprises an amino acid sequence having at least 70%, preferably at least 75%, preferably at least 80%, preferably at least 85%, preferably at least 90%, preferably at least 95%, or more preferably at least 99% sequence identity to SEQ ID NO:1.

Illustrative examples of suitable structural analogues of SEQ ID NO:1 are described in WO2019/136528, the entire contents of which is incorporated herein by reference. In an embodiment, the structural analogue comprises, consists or consists essentially of the peptide of formula (I):

(SEQ ID NO: 3)
(I)
R¹-CRSVEGSCG-R²

wherein
R¹ is selected from the group consisting of YLRIVQ (SEQ ID NO:4), LRIVQ (SEQ ID NO:5), RIVQ (SEQ ID NO:6), IVQ (SEQ ID NO:7), VQ (SEQ ID NO:8), and Q (SEQ ID NO:9), or R¹ is absent; and
R² is F (phenylalanine; SEQ ID NO:10), or R² is absent, wherein the peptide of formula (I) is a cyclic peptide formed by a disulphide bond between the two cysteine residues.

In an embodiment, the structural analogue comprises, consists, or consists essentially of an amino acid sequence selected from the group consisting of LRIVQCRSVEGSCGF (SEQ ID NO:11), CRSVEGSCG (SEQ ID NO:12; LAT9991), CRSVEGSCGF (SEQ ID NO:13; LAT9991F) and an amino acid sequence having at least 70%, preferably at least 75%, preferably at least 80%, preferably at least 85%, preferably at least 90%, preferably at least 95%, or more preferably at least 99% sequence identity to any of the foregoing.

In an embodiment, the structural analogue comprises, consists, or consists essentially of the a peptide of formula (II), or a pharmaceutically acceptable salt thereof:

(SEQ ID NO: 14)
(II)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is alanine, arginine or lysine;
X⁴ is glutamic acid or aspartic acid;
R¹ is selected from the group consisting of:

```
  (SEQ ID NO: 15)
S,
  (SEQ ID NO: 16)
HS,
  (SEQ ID NO: 17)
GHS,
  (SEQ ID NO: 18)
PGHS,
  (SEQ ID NO: 19)
APGHS,
  (SEQ ID NO: 20)
EAPGHS,
  (SEQ ID NO: 21)
SEAPGHS,
  (SEQ ID NO: 22)
SSEAPGHS,
  (SEQ ID NO: 23)
PSSEAPGHS,
  (SEQ ID NO: 24)
DPSSEAPGHS,
and
  (SEQ ID NO: 25)
IDPSSEAPGHS,
```

or R¹ is absent; and
R² is selected from the group consisting of:

```
  (SEQ ID NO: 26)
S,
  (SEQ ID NO: 27)
SS,
(SEQ ID NO: 28)
SSK,
(SEQ ID NO: 29)
SSKF,
(SEQ ID NO: 30)
SSKFS,
  (SEQ ID NO: 31)
SSKFSW,
  (SEQ ID NO: 32)
SSKFSWD,
  (SEQ ID NO: 33)
SSKFSWDE,
  (SEQ ID NO: 34)
SSKFSWDEY,
  (SEQ ID NO: 35)
SSKFSWDEYE,
  (SEQ ID NO: 36)
SSKFSWDEYEQ,
  (SEQ ID NO: 37)
SSKFSWDEYEQY,
  (SEQ ID NO: 38)
SSKFSWDEYEQYK,
  (SEQ ID NO: 39)
SSKFSWDEYEQYKK,
and
  (SEQ ID NO: 40)
SSKFSWDEYEQYKKE,
```

or R² is absent; and
wherein the peptide of formula (II) is a cyclic peptide formed by a disulphide bond between the two cysteine residues.

In an embodiment, the structural analogue comprises, consists, or consists essentially of an amino acid sequence selected from the group consisting of SCRSRPVESSC (SEQ ID NO:41; LAT9993), CRSRPVESSC (SEQ ID NO:42), CRSRPVESSCS (SEQ ID NO:43), SCRSRPVESSCS (SEQ ID NO:44; LAT9993S) and an amino acid sequence having at least 70%, preferably at least 75%, preferably at least 80%, preferably at least 85%, preferably at least 90%, preferably at least 95%, or more preferably at least 99% sequence identity to any of the foregoing.

In another embodiment, the structural analogue comprises, consists, or consists essentially of the peptide of formula (III):

(SEQ ID NO:45)
(III)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:46), LKLLK (SEQ ID NO:47), KLLK (SEQ ID NO:48), LLK (SEQ ID NO:49), LL (SEQ ID NO:50), K (SEQ ID NO:51) or R¹ is absent; and
R² is G (glycine), or R² is absent
wherein the peptide of formula (III) is a cyclic peptide formed by a disulphide bond between the two cysteine residues.

In an embodiment, the structural analogue comprises, consists, or consists essentially of an amino acid sequence selected from the group consisting of CRIIHNNNC (SEQ ID NO:52; LAT7771), CRIIHNNNCG (SEQ ID NO:53), CRIVYDSNC (SEQ ID NO:54), CRIVYDSNCG (SEQ ID NO:55) and an amino acid sequence having at least 70%, preferably at least 75%, preferably at least 80%, preferably at least 85%, preferably at least 90%, preferably at least 95%, or more preferably at least 99% sequence identity to any of the foregoing.

In an embodiment, the structural analogue of the cyclic peptide comprising SEQ ID NO:1 is derived from human interleukin-1 receptor-associated kinase 3 (IRAK-3).

In an embodiment, the structural analogue of the cyclic peptide comprising SEQ ID NO:1 is derived from human prolactin.

The present disclosure also extends to isolating and/or producing the analgesic agents identified by the methods disclosed herein. Thus, in an embodiment, the method further comprises isolating, synthesizing or otherwise producing the candidate agent identified as an analgesic agent according to the methods disclosed herein.

As described elsewhere herein, functional variants and structural analogues, as herein described, include molecules that have an amino acid sequence that differs from the amino acid sequence of native protein by one or more (e.g., 1, 2, 3, 4, 5, 6, 7, 8, 9 or more) amino acid insertions, deletions and/or substitutions. In some embodiments, functional variants and structural analogues will have an amino acid sequence that differs from the amino acid sequence of native protein by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, functional variants and structural analogues will have at least 85% sequence identity to an amino acid sequence of native protein. Reference to “at least 85%” includes 85%, 86%, 87%, 88%, 89%, 90%, 91%, 92%, 93%, 94%, 95%, 96%, 97%, 98%, 99% or 100% sequence identity or similarity, for example, after optimal alignment or best fit analysis. Thus, in an embodiment, the sequence has at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity or sequence homology with the sequences identified herein, for example, after optimal alignment or best fit analysis.

The terms “identity”, “similarity”, “sequence identity”, “sequence similarity”, “homology”, “sequence homology” and the like, as used herein, mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. As noted elsewhere herein, this may be referred to as conservative substitution. In an embodiment, an amino acid sequence may be modified by way of conservative substitution of any of the amino acid residues contained therein, such that the modification has no effect on the ability of the functional variant to alleviate pain when administered to a subject in need thereof when compared to the unmodified (native) peptide/protein.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff (Atlas of Protein Sequences and Structure, M. O. Dayhoff ed., 5 suppl. 3:353-358, National Biomedical Research Foundation, Washington, D.C., USA), and normalized by Gribskov (Nucl. Acids Res. 14(6):6745-6763, 1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., (1997, Nucl. Acids Res. 25:3389). A detailed discussion of sequence analysis can be found in Unit 19.3 of Ausubel et al. (“Current Protocols in Molecular Biology”, John Wiley & Sons Inc, 1994-1998, Chapter 15).

“Similarity” refers to the percentage number of amino acids that are identical or constitute conservative substitutions as defined in Tables 1 and 2 supra. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

### Methods of Treatment

As noted elsewhere herein, the present invention is predicated, at least in part, on the inventor's identification and characterisation of the molecular target of a new class of cyclic peptide molecules to which analgesic properties have recently been ascribed. This is the first time that the molecular target of this new class of analgesic peptides has been identified and characterised, allowing for the identification and/or design of new therapeutic agents for the treatment of pain, including neuropathic pain. Accordingly, in an aspect disclosed herein, there is provided a method of treating pain in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a peptide comprising SEQ ID NO:1 (YLRIVQCRSVEGSCGF).

As persons skilled in the art will know, there are many possible causes of pain. It is therefore to be understood that contemplated herein is the treatment or prevention of pain regardless of cause. In some embodiments, pain is a result of injury or trauma to tissue, disease or condition affecting the nerves (e.g., primary neuropathy) and/or pain that is caused by systemic disease (secondary neuropathy), illustrative examples of which include diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathies; hereditary motor and sensory neuropathies (HMSN); hereditary sensory neuropathies (HSNs); hereditary sensory and autonomic neuropathies; hereditary neuropathies with ulcero-mutilation; nitrofurantoin neuropathy; tomaculous neuropathy; neuropathy caused by nutritional deficiency, neuropathy caused by kidney failure and complex regional pain syndrome. Other illustrative examples of conditions that may cause pain include repetitive activities such as typing or working on an assembly line, medications known to cause peripheral neuropathy such as several antiretroviral drugs ddC (zalcitabine) and ddI (didanosine), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated with infectious processes (such as Guillain-Barré syndrome). Other illustrative examples of neuropathic pain include thermal or mechanical hyperalgesia, thermal or mechanical allodynia, diabetic pain, neuropathic pain affecting the oral cavity (e.g., trigeminal neuropathic pain, atypical odontalgia (phantom tooth pain), burning mouth syndrome), fibromyalgia and entrapment pain.

In an embodiment disclosed herein, the pain is selected from the group consisting of cancer pain; toothaches; visceral pain such as pancreatitis; pelvic pain; thermal and chemical burns; pain associated with inflammation or bacterial, fungal or viral infections; pain associated with metabolic diseases, anorexia, obesity and obesity related conditions, pain associated with impaired glucose tolerance and diabetes; pain associated with osteoarthritis; pain associated with musculoskeletal and neuromuscular diseases; pain associated with conditions with impaired chondrocyte, proteoglycan or collagen production or quality; pain associated with conditions with impaired cartilage tissue formation or quality; pain associated with conditions with impaired muscle, ligament or tendon mass, form or function; pain associated with inflammation, trauma or a genetic abnormality affecting muscle or connective tissue and bone disorders; pain associated with muscular dystrophy, pain associated with AIDS wasting syndrome, pain associated with Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, allodynia, hyperesthesia, hyperalgesia, burning pain and shooting pain, drug-induced peripheral neuropathy (e.g., chemo-therapy induced neuropathy), and peripheral neuropathy associated with infection.

In some embodiments, the pain may be accompanied by numbness, weakness and loss of reflexes. The pain may be severe and disabling. By “hyperalgesia” is meant an increased response to a stimulus that is normally painful. A hyperalgesia condition is one that is associated with pain caused by a stimulus that is not normally painful. The term “hyperesthesia” refers to an excessive physical sensitivity, especially of the skin. The term “allodynia” as used herein refers to the pain that results from a non-noxious stimulus; that is, pain due to a stimulus that does not normally provoke pain. Illustrative examples of allodynia include thermal allodynia (pain due to a cold or hot stimulus), tactile allodynia (pain due to light pressure or touch), mechanical allodynia (pain due to heavy pressure or pinprick) and the like.

Pain may be acute or chronic and, in this context, it is to be understood that the time course of a pain may vary, based on its underlying cause. For instance, with trauma, the onset of symptoms of pain may be acute, or sudden; however, the most severe symptoms may develop over time and persist for years. A chronic time course over weeks to months usually indicates a toxic or metabolic pain syndrome. A chronic, slowly progressive pain syndrome, such as occurs with painful diabetic neuropathy or with most hereditary neuropathies or with a condition termed chronic inflammatory demyelinating polyradiculoneuropathy (CIDP), may have a time course over many years. Neuropathic conditions with symptoms that relapse and remit include Guillain-Barré syndrome.

In some embodiments, neuropathic pain results from a condition characterised by neuronal hypersensitivity, such as fibromyalgia or irritable bowel syndrome.

In other embodiments, neuropathic pain results from a disorder associated with aberrant nerve regeneration resulting in neuronal hypersensitivity. Such disorders include breast pain, interstitial cystitis, vulvodynia and cancer chemotherapy-induced neuropathy.

In some embodiments, the pain is related to surgery, pre-operative pain and post-operative pain, particularly post-operative pain.

In some embodiments, the pain is related to trauma or injury to tissue, chemical or thermal burns, or visceral pain.

In an embodiment, the pain is neuropathic pain. Without being bound by theory, or by a particular mode of application, neuropathic pain is typically characterised as pain which results from damage by injury or disease to nerve tissue or neurones per se or of dysfunction within nerve tissue. The pain may be peripheral, central or a combination thereof; in other words, the term “neuropathic pain” typically refers to any pain syndrome initiated or caused by a primary lesion or dysfunction in the peripheral or central nervous system. Neuropathic pain is also distinguishable in that it typically does not respond effectively to treatment by common pain medication such as opioids. By contrast, nociceptive pain is characterised as pain which results from stimulation of nociceptors by noxious or potentially harmful stimuli that may cause damage or injury to tissue. Nociceptive pain is typically responsive to common pain medication, such as opioids.

Types of neuropathic pain will be familiar to persons skilled in the art, illustrative examples of which include diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, migraine, drug-induced peripheral neuropathy (e.g., chemo-therapy induced neuropathy) and peripheral neuropathy associated with infection, chronic low back pain, complex regional pain syndrome, temporomandibular joint disorders, Lichen Planus and reflex sympathetic dystrophy.

Thus, in an embodiment, the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, migraine, drug-induced peripheral neuropathy (e.g., chemo-therapy induced neuropathy) and peripheral neuropathy associated with infection, chronic low back pain, complex regional pain syndrome, temporomandibular joint disorders, Lichen Planus and reflex sympathetic dystrophy.

In an embodiment, the agent is not a cyclic peptide derived from human interleukin-1 receptor-associated kinase 3 (IRAK-3).

In an embodiment, the agent is not a cyclic peptide derived from human prolactin.

The present disclosure also extends to adjunct therapies. Thus, in an embodiment, the method further comprises administering to the subject in need thereof an additional analgesic agent, wherein the additional analgesic agent is not (i) an agent that binds to LANCL1, or (ii) an agent that competes for binding to LANCL1 with a cyclic peptide comprising SEQ ID NO: 1. Suitable analgesic agents will be familiar to persons skilled in the art and may include agents that treat nociceptive pain and/or neuropathic pain. Illustrative examples of suitable analgesic agents include morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID). Illustrative examples of suitable NSAIDs include aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelonean.

In an embodiment, the additional analgesic agent comprises an agent capable of alleviating nociceptive pain in a subject. In an embodiment, the additional analgesic agent is selected from the group consisting of morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID). In an embodiment, the additional analgesic agent is a non-steroidal anti-inflammatory drug (NSAID). In a preferred embodiment, the NSAID is selected from the group consisting of aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelonean. In an embodiment, the additional analgesic agent is not an agent that binds to LANCL1.

The terms “treating”, “treatment” and the like, are used interchangeably herein to mean relieving, reducing, alleviating, ameliorating or otherwise inhibiting pain, including one or more symptoms of pain, such as allodynia or hyperalgesia. The terms “prevent”, “preventing”, “prophylaxis”, “prophylactic”, “preventative” and the like are used interchangeably herein to mean preventing or delaying the onset of pain, or the risk of developing pain.

The terms “treating”, “treatment” and the like also include relieving, reducing, alleviating, ameliorating or otherwise inhibiting the effects of the pain for at least a period of time. It is also to be understood that terms “treating”, “treatment” and the like do not imply that the pain, or a symptom thereof, is permanently relieved, reduced, alleviated, ameliorated or otherwise inhibited and therefore also encompasses the temporary relief, reduction, alleviation, amelioration or otherwise inhibition of pain, or a symptom thereof.

As noted elsewhere herein, the present invention is predicated, at least in part, on the inventor's identification and characterisation of the molecular target of a new class of cyclic peptide molecules to which therapeutic (other than analgaesic) properties have been ascribed. Accordingly, in another aspect disclosed herein, there is provided a method of treating a condition in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising SEQ ID NO:1 (YLRIVQCRSVEGSCGF), wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

In an embodiment, the agent is not a peptide derived from an interleukin-1 receptor-associated kinase 3 (IRAK-3). In an embodiment, the agent is not a peptide derived from human prolactin. In an embodiment, the condition is a respiratory condition. Illustrative examples of respiratory conditions include chronic obstructive pulmonary disease, asthma, cystic fibrosis and lung cancer and respiratory tract infection. Thus, in an embodiment, the respiratory condition is selected from the group consisting of chronic obstructive pulmonary disease, asthma, cystic fibrosis and lung cancer and a respiratory tract infection. In an embodiment, the respiratory condition is a respiratory tract infection.

Respiratory tract infection (RTI) is typically defined as any infectious disease of the upper or lower respiratory tract. Upper respiratory tract infections (URTIs) include the common cold, laryngitis, pharyngitis/tonsillitis, acute rhinitis, acute rhinosinusitis and acute otitis media. Lower respiratory tract infections (LRTIs) include acute bronchitis, bronchiolitis, pneumonia and tracheitis. Antibiotics are commonly prescribed for RTIs in adults and children in primary care. RTIs are the reason for 60% of all antibiotic prescribing in general practice, and this constitutes a significant cost to the health system (NICE Clinical Guidelines, No. 69; Centre for Clinical Practice at NICE (UK), London: National Institute for Health and Clinical Excellence (UK); 2008).

Pathogens that give rise to infection of the upper and/or lower respiratory tracts in human and non-human subjects will be known to persons skilled in the art, and include bacteria and viruses, illustrative examples of which are described in Charlton et al. (Clinical Microbiology Reviews; 2018, 32 (1): e00042-18), Popescu et al. (Microorganisms. 2019; 7(11): 521) and Kikkert, M. (J. Innate Immun. 2020; 12(1): 4-20), the contents of which are incorporated herein by reference in their entirety. In an embodiment, the respiratory tract infection is a virus infection.

Viruses that give rise to infection of the respiratory tract in human and non-human subjects (upper and/or lower respiratory tracts) will be known to persons skilled in the art, illustrative examples of which include a picornavirus, a coronavirus, an influenza virus, a parainfluenza virus, a respiratory syncytial virus, an adenovirus, an enterovirus, and a metapneumovirus. Thus, in an embodiment disclosed herein, the virus is selected from the group consisting of a picornavirus, a coronavirus, an influenza virus, a parainfluenza virus, a respiratory syncytial virus, an adenovirus, an enterovirus, and a metapneumovirus. In an embodiment, the virus is an influenza virus. In another embodiment, the virus is a coronavirus. Illustrative examples of coronaviruses that give rise to respiratory tract infection will be familiar to persons skilled in the art, illustrative examples of which include SARS-CoV-2 as previously described in Zhu N et al., (2019. N Engl J Med. 2020) and in US patent publication no. 20190389816, the contents of which are incorporated herein by reference in their entirety. In an embodiment, the virus is SARS-CoV-2.

The methods, compositions and uses thereof, as described herein, may be particularly useful for treating a respiratory tract infection, including in subjects with an underlying medical condition that would otherwise exacerbate the respiratory tract infection. Such underlying conditions will be known to persons skilled in the art, illustrative examples of which include chronic obstructive pulmonary disease, asthma, cystic fibrosis, emphysema and lung cancer. In an embodiment, the condition is selected from the group consisting of chronic obstructive pulmonary disease, asthma, cystic fibrosis, respiratory tract infection, lung cancer and a combination of any of the foregoing. In another embodiment, the subject is immunocompromised, whether as a result of treatment (e.g., by chemotherapy, radiotherapy) or otherwise (e.g., by HIV infection). In an embodiment, the respiratory tract infection is a virus infection

As used herein, the term “administered” refers to the placement of an agent described herein, into a subject by a method or route which results in at least partial localization of the compound at a desired site. An agent described herein can be administered by any appropriate route which results in effective treatment in the subject, i.e., administration results in delivery to a desired location in the subject where at least a portion of the composition delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle. As used herein, the term “condition” includes anatomic and physiological deviations from the normal that constitute an impairment of the normal state of the living animal or one of its parts, that interrupts or modifies the performance of the bodily functions.

The terms “decrease”, “reduce” or “inhibit” and their grammatical equivalents are all used herein generally to mean a decrease by a statistically significant amount. However, for avoidance of doubt, the terms “decrease”, “reduce” or “inhibit” and their grammatical equivalents mean a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increase”, “enhance”, or “activate” and their grammatical equivalents are all used herein to generally mean an increase by a statically significant amount; for the avoidance of any doubt, the terms “increase”, “enhance”, or “activate” and their grammatical equivalents mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The phrase “therapeutically effective amount” typically means an amount necessary to attain the desired response, including to delay the onset or inhibit progression or halt altogether, the onset or progression of pain being treated. It would be understood by persons skilled in the art that the therapeutically effective amount of peptide will vary depending upon several factors, illustrative examples of which include the health and physical condition of the subject to be treated, the taxonomic group of subject to be treated, the severity of the pain to be treated, the formulation of the composition to be administered, the route of administration, and combinations of any of the foregoing.

The therapeutically effective amount will typically fall within a relatively broad range that can be determined through routine trials by persons skilled in the art. Illustrative examples of a suitable therapeutically effective amount of the therapeutic agent identified by the methods disclosed herein for administration to a subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. In an embodiment disclosed herein, the therapeutically effective amount is from about 0.001 mg per kg of body weight to about 1 g per kg of body weight per dose (e.g., 0.001 mg/kg, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg of body weight, etc). In an embodiment, the therapeutically effective amount is from about 0.001 mg to about 50 mg per kg of body weight. In an embodiment, the therapeutically effective amount is from about 0.01 mg to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation

The term “analgesia” is used herein to describe states of reduced pain perception, including absence from pain sensations, as well as states of reduced or absent sensitivity to noxious stimuli. Such states of reduced or absent pain perception are typically induced by the administration of a pain-controlling agent or agents and occur without loss of consciousness, as is commonly understood in the art. Suitable methods for determining whether a compound is capable of providing an analgesic effect will be familiar to persons skilled in the art, illustrative examples of which include the use of animal models of neuropathic pain, such as chronic constriction injury, spinal nerve ligation and partial sciatic nerve ligation (see Bennett et al. (2003); Curr. Protoc. Neurosci., Chapter 9, Unit 9.14) and animal models of nociceptive pain, such as formalin-, carrageenan- or complete Freund's adjuvant (CFA)-induced inflammatory pain. Other suitable models of pain are discussed in Gregory et al. (2013, J. Pain.; 14(11); “An overview of animal models of pain: disease models and outcome measures”).

The term “subject”, as used herein, refers to a mammalian subject for whom treatment or prophylaxis of pain is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In another embodiment, the subject is selected from the group consisting of a canine, a feline and an equine.

It is to be understood that a reference to a subject herein does not imply that the subject has pain, or a symptom thereof, but also includes a subject that is at risk of developing pain, or a symptom thereof. In an embodiment, the subject has (i.e., is experiencing) pain or a symptom thereof. In another embodiment, the subject is not experiencing pain or a symptom thereof at the time of treatment, but is at risk of developing pain or a symptom thereof. In an illustrative example, the subject has a disease or condition that puts the subject at risk of developing pain, for example, poorly managed diabetes, which may lead to a diabetic neuropathy. In another embodiment, the subject has had a disease or condition that has potential to result in pain, such as herpes zoster (shingles), which may lead to post-herpetic neuralgia.

In an embodiment, the pain is a migraine. Migraine typically features episodic, recurrent disabling headaches lasting between 4-72 hours, which may be accompanied by other symptoms, such as nausea, vomiting, phonophobia, photophobia, speech disturbances and visual auras. A migraine attack will typically have four phases: 1) the premonitory stage, occurring several hours before a headache, and characterized by symptoms such as fatigue, irritability, difficulty concentrating, mood change, yawning, stiff neck, phonophobia, and/or nausea; 2) the aura phase, with symptoms of sensory or cognitive disturbance; 3) the headache phase comprising throbbing pain, nausea, vomiting and sensory sensitivity; and 4) the postdrome phase, occurring hours to days after resolution of the headache, with symptoms such as weakness, cognitive difficulties, mood changes and gastrointestinal symptoms.

Migraine may be episodic (acute) or chronic. As defined by The International Classification of Headache Disorders (3^{rd} edition, The International Headache Society, 2018), when migraine occurs on fewer than 15 days per month, it is considered episodic, whereas chronic migraine is typically defined as more than 15 headache days per month over a three month period, of which more than eight are migrainous.

It is to be understood that contemplated herein is the treatment or prevention of migraine regardless of cause. In some embodiments, the migraine is accompanied by numbness, weakness and/or loss of reflexes. In some embodiments, the migraine is accompanied by severe and/or disabling pain. It is to be understood that a reference to a subject herein does not imply that the subject has migraine, or a symptom thereof, but also includes a subject that is at risk of developing migraine, or a symptom thereof. In an embodiment, the subject has (i.e., is experiencing) migraine or a symptom thereof. In another embodiment, the subject is not experiencing migraine or a symptom thereof at the time of treatment, but is at risk of developing migraine or a symptom thereof. In an embodiment, the subject suffers from chronic migraine. In another embodiment, the subject suffers from episodic (acute) migraine.

The therapeutic agents disclosed herein (i.e., as capable of binding to LANCL1 and competing for binding with a cyclic peptide comprising SEQ ID NO:1, or a structural homologue thereof) may be administered to the subject by any suitable route that allows for delivery of the peptides to the subject at a therapeutically effective amount, as herein described. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation). The therapeutic agents disclosed herein may also suitably be administered to the subject as a controlled release dosage form to provide a controlled release of the active agent(s) over an extended period of time. The term “controlled release” typically means the release of the active agent(s) to provide a constant, or substantially constant, concentration of the active agent in the subject over a period of time (e.g., about eight hours up to about 12 hours, up to about 14 hours, up to about 16 hours, up to about 18 hours, up to about 20 hours, up to a day, up to a week, up to a month, or more than a month). Controlled release of the active agent(s) can begin within a few minutes after administration or after expiration of a delay period (lag time) after administration, as may be required. Suitable controlled release dosage forms will be known to persons skilled in the art, illustrative examples of which are described in Anal, A. K. (2010; Controlled-Release Dosage Forms. Pharmaceutical Sciences Encyclopedia. 11:1-46).

Without being bound by theory or by a particular mode of application, it may be desirable to elect a route of administration on the basis of whether the pain is localized or generalised. For example, where the pain is localized, it may be desirable to administer the agents to the affected area or to an area immediately adjacent thereto. For instance, where the pain is in a joint (e.g., neck, knee, elbow, shoulder, hip, etc.), the agents can be administered to the subject intra-articularly into the affected joint. Alternatively, or in addition, the agents can be administered at, or substantially adjacent to, the affected joint. As another illustrative example, where the pain is in the oral cavity (e.g., trigeminal neuropathic pain, atypical odontalgia (phantom tooth pain) or burning mouth syndrome), the agents can be formulated for administration via the oral mucosa (e.g., by buccal and/or sublingual administration). Conversely, where the pain is generalised or disseminated across multiple anatomical sites of a subject, the agents may be administered topically, enterally and/or parenterally at any site with a view to distributing the agents across the multiple anatomical sites affected by neuropathic pain. In an embodiment disclosed herein, the agents disclosed herein are administered to the subject enterally. In an embodiment disclosed herein, the agents disclosed herein are administered to the subject orally. In an embodiment disclosed herein, the agents disclosed herein are administered to the subject parenterally. In another embodiment disclosed herein, the agents disclosed herein are administered to the subject topically. As described elsewhere herein, “topical” administration typically means application of the agents to a surface of the body, such as the skin or mucous membranes, suitably in the form of a cream, lotion, foam, gel, ointment, nasal drop, eye drop, ear drop, transdermal patch, transdermal film (e.g., sublingual film) and the like. Topical administration also encompasses administration via the mucosal membrane of the respiratory tract by inhalation or insufflation. In an embodiment disclosed herein, the topical administration is selected from the group consisting of transdermal and transmucosal administration. In an embodiment, the peptides disclosed herein are administered to the subject transdermally.

In an embodiment, the methods comprise orally administering the agents disclosed herein to a human. In another embodiment, the methods comprise orally administering the agents disclosed herein to a non-human subject. In yet another embodiment, the methods comprise orally administering the agents disclosed herein to a non-human subject selected from the group consisting of a feline, a canine and an equine.

In an embodiment, the methods comprise administering the agents disclosed herein topically to a human. In another embodiment, the methods comprise administering the agents disclosed herein topically to a non-human subject. In yet another embodiment, the methods comprise administering the agents disclosed herein topically to a non-human subject selected from the group consisting of a feline, a canine and an equine.

In an embodiment disclosed herein, the agents disclosed herein are administered to the subject as a controlled release dosage form, illustrative examples of which are described elsewhere herein. In an embodiment, the methods comprise administering the agents disclosed herein to a human as a controlled release dosage form. In another embodiment, the methods comprise administering the agents disclosed herein to a non-human subject as a controlled release dosage form. In yet another embodiment, the methods comprise administering the agents disclosed herein as a controlled release dosage form to a non-human subject selected from the group consisting of a feline, a canine and an equine.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, it may be beneficial to administer the agents, as herein disclosed, via more than one route. For example, it may be desirable to administer a first dose parenterally (e.g., via intramuscular, intravenous; subcutaneous, epidural, intra-articular, intraperitoneal, intracisternal or intrathecal routes of administration) to induce a rapid or otherwise acute analgesic effect in a subject, followed by a subsequent (e.g., second, third, fourth, fifth, etc) dose administered enterally (e.g., orally or rectally) and/or topically (e.g., via transdermal or transmucosal routes of administration) to provide continuing availability of the active agent over an extended period subsequent to the acute phase of treatment. Alternatively, it may be desirable to administer a dose enterally (e.g., orally or rectally), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or topically (e.g., via transdermal or transmucosal routes of administration). Alternatively, it may be desirable to administer a dose topically (e.g., via transdermal or transmucosal routes of administration), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or enterally (e.g., orally or rectally).

The route of administration may suitably be selected on the basis of whether the pain is localised or generalised, as discussed elsewhere herein. Alternatively, or in addition, the route of administration may suitably be selected having regard to factors such as the subject's general health, age, weight and tolerance (or a lack thereof) for given routes of administration (e.g., where there is a phobia of needles, an alternative route of administration may be selected, such as enteral and/or topical).

It is also to be understood that, where multiple routes of administration are desired, any combination of two or more routes of administration may be used in accordance with the methods disclosed herein. Illustrative examples of suitable combinations include, but are not limited to, (in order of administration), (a) parenteral-enteral; (b) parenteral-topical; (c) parenteral-enteral-topical; (d) parenteral-topical-enteral; (e) enteral-parenteral; (f) enteral-topical; (g) enteral-topical-parenteral; (h) enteral-parenteral-topical; (i) topical-parenteral; (j) topical-enteral; (k) topical-parenteral-enteral; (l) topical-enteral-parenteral; (m) parenteral-enteral-topical-parenteral; (n) parenteral-enteral-topical-enteral; etc.

In an embodiment, the methods comprise (i) parenterally administering to the subject the agents disclosed herein, and (ii) non-parenterally (i.e, enterally or topically) administering to the subject the agents disclosed herein, wherein the non-parenteral (enteral or topical) administration is subsequent to the parenteral administration. In an embodiment, the parenteral administration is selected from the group consisting of intramuscular, a subcutaneous and intravenous. In a further embodiment, the parenteral administration is subcutaneous. In an embodiment, the non-parenteral administration is oral.

In an embodiment, the methods disclosed herein comprise (i) parenterally administering to a human subject the agents disclosed herein and (ii) orally administering to the human subject the agents disclosed herein, wherein the oral administration is subsequent to the parenteral administration. In an embodiment, the parenteral administration is subcutaneous. In another embodiment, the parenteral administration is intrathecal.

In an embodiment, the methods disclosed herein comprise (i) parenterally administering to a non-human subject the agents disclosed herein, and (ii) orally administering to the non-human subject the peptides disclosed herein, wherein the oral administration is subsequent to the parenteral administration.

In an embodiment, the non-human subject is selected from the group consisting of a feline, a canine and an equine. In an embodiment, the parenteral administration is subcutaneous. In another embodiment, the parenteral administration is intrathecal.

In another embodiment, the methods disclosed herein comprise (i) parenterally administering to a human subject the agents disclosed herein, and (ii) topically administering to the human subject the agents disclosed herein, wherein the topical administration is subsequent to the parenteral administration.

In a further embodiment, the methods disclosed herein comprise (i) parenterally administering to a non-human subject the agents disclosed herein, and (ii) topically administering to the non-human subject the agents disclosed herein, wherein the topical administration is subsequent to the parenteral administration. In an embodiment, the non-human subject is selected from the group consisting of a feline, a canine and an equine. In an embodiment, the parenteral route of administration is subcutaneous. In another embodiment, the topical route of administration is transdermal. In another embodiment, the parenteral administration is subcutaneous and the topical administration is transdermal.

Alternatively, or in addition, the agents disclosed herein may suitably be administered as a controlled release dosage form. Thus, in an embodiment, the methods comprise (i) parenterally administering to the subject the agents disclosed herein, and (ii) administering to the subject the agents disclosed herein, as a controlled release dosage form, wherein the controlled release dosage form is administered subsequent to the parenteral administration. In another embodiment, the methods comprise (i) non-parenterally (enterally or topically) administering to the subject the agents disclosed herein, and (ii) administering to the subject the agents disclosed herein, as a controlled release dosage form, wherein the controlled release dosage form is administered to the subject subsequent to the non-parenteral administration. In yet another embodiment, the methods comprise (i) enterally administering to the subject the agents disclosed herein, and (ii) administering to the subject the agents disclosed herein, as a controlled release dosage form, wherein the controlled release dosage form is administered to the subject subsequent to the enteral administration. In yet another embodiment, the methods comprise (i) topically administering to the subject the agents disclosed herein, and (ii) administering to the subject the agents disclosed herein, as a controlled release dosage form, wherein the controlled release dosage form is administered to the subject subsequent to the topical administration. In a preferred embodiment, the controlled release dosage form is formulated for parenteral administration.

As noted elsewhere herein, the therapeutic agents identified by the methods disclosed herein may suitably be administered together, either sequentially or in combination (e.g., as an admixture), with one or more additional active agents. It will be understood by persons skilled in the art that the nature of the other active agents will depend on the condition to be treated or prevented. For example, where the subject has cancer, the therapeutic agents disclosed herein may be administered to the subject together, either sequentially or in combination (e.g., as an admixture), with one or more chemotherapeutic agents, illustrative examples of which will be familiar to persons skilled in the art. Combination treatments of this nature can be advantageous by alleviating the pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide. Thus, in an embodiment, the methods disclosed herein further comprise administering to the subject a therapeutically effective amount of a chemotherapeutic agent.

The agents disclosed herein may also be suitably administered to the subject together, either sequentially or in combination (e.g., as an admixture), with one or more additional analgesic agents capable of alleviating pain in the subject. Suitable additional analgesic agents will be familiar to persons skilled in the art, illustrative examples of which include analgesic agents capable of alleviating nociceptive pain, agents capable of alleviating neuropathic pain, or any combination thereof. Thus, in an embodiment, the methods disclosed herein further comprise administering to the subject a therapeutically effective amount of an additional analgesic agent capable of alleviating pain in the subject.

In an embodiment, the additional analgesic agent is capable of alleviating nociceptive pain in the subject. In another embodiment, the additional analgesic agent is capable of alleviating neuropathic pain in the subject.

Suitable agents capable of alleviating nociceptive pain will be familiar to persons skilled in the art, illustrative examples of which are described elsewhere herein and include opiates such as morphine, fentanyl, tramadol, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the additional analgesic agent capable of alleviating nociceptive pain is an opioid. In an embodiment, the additional analgesic agent is an NSAID

In other embodiments disclosed herein, the agents disclosed herein are administered together, either sequentially or in combination (e.g., as an admixture), with another therapy to treat or alleviate neuropathic pain or the underlying condition that is causing the neuropathic pain. In some instances, the amount of the additional analgesic agent may be reduced when administration is together with the peptides disclosed herein. Illustrative examples of suitable agents capable of treating neuropathic pain include duloxetine, pregabalin, gabapentin, phenytoin, melatonin, carbamazepine, levocarnitine, capsaicin, tricyclic antidepressants such as amitryptiline and sodium channel blockers such as lidocaine

Also disclosed herein is a composition for use in treating a condition in a subject in need thereof, the composition comprising an agent that binds to Lanthionine synthetase C-like protein 1 (LANCL1), and competes for binding to LANCL1 with a cyclic peptide comprising SEQ ID NO:1, or with a structural analogue thereof, and wherein the agent is not a peptide derived from human growth hormone or from a non-human homologue thereof, wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

The present disclosure also extends to use of an agent that binds to Lanthionine synthetase C-like protein 1 (LANCL1) and competes for binding to LANCL1 with a cyclic peptide of SEQ ID NO:1, or with a structural analogue thereof, in the manufacture of a medicament for treating a condition in a subject in need thereof, wherein the agent is not a peptide derived from human growth hormone or from a non-human homologue thereof and wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

### Pharmaceutical Compositions

The therapeutic agents disclosed herein may be formulated for administration to a subject as a neat chemical or compound. However, in certain embodiments, it may be preferable to formulate the agents disclosed herein as a pharmaceutical composition, including veterinary compositions. Thus, also disclosed herein is a composition comprising the agent identified by the screening methods disclosed herein, wherein the agent is not a peptide derived from human growth hormone or from a non-human homologue thereof.

The agent identified according to the methods disclosed herein will suitably be (i) capable of binding to LANCL1, and (ii) capable of competing for binding to LANCL1, with a cyclic peptide of SEQ ID NO:1, or with a structural analogue thereof, wherein the agent is not a peptide derived from human growth hormone or from a non-human homologue thereof.

In an embodiment, the composition further comprises a pharmaceutically acceptable carrier. In an embodiment, the composition further comprises a pharmaceutically acceptable carrier, excipient or diluent.

As used herein, the term “pharmaceutically acceptable” refers to those compounds, agents, materials, compositions, and/or dosage forms which are, within the scope of sound medical judgment, suitable for administration to a subject without excessive toxicity, irritation, allergic response, or other problem or complication, commensurate with a reasonable benefit/risk ratio.

As used herein, the term “pharmaceutically-acceptable carrier” typically means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the patient.

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions disclosed herein may also contain additional ingredients and/or materials commonly used in pharmaceutical compositions, including therapeutic antigen-binding molecule preparations. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

In an embodiment, the agents disclosed herein are formulated for administration to the subject in a therapeutically effective amount that alleviates pain or any other condition to be treated in the subject, as described elsewhere herein.

In an embodiment, the agents are formulated for administration sequentially, or in combination, with an additional analgesic agent capable of alleviating pain in the subject. In an embodiment, the agents are formulated for administration sequentially, or in combination, with an additional therapeutic agent capable of treating a condition in the subject, as herein described. In an embodiment, the additional analgesic agent is capable of alleviating nociceptive pain in the subject, illustrative examples of which are described elsewhere herein. In another embodiment, the additional analgesic agent is capable of alleviating neuropathic pain in the subject, illustrative examples of which are also described elsewhere herein. In an embodiment, the additional analgesic agent is an opioid.

The agents disclosed herein may be administered together, either sequentially or in combination (e.g., as an admixture), with one or more another active agents that will likely depend on the condition to be treated. For example, where the subject has cancer, the compositions disclosed herein may be formulated for administration together, either sequentially or in combination (e.g., as an admixture), with one or more chemotherapeutic agents, illustrative examples of which will be familiar to persons skilled in the art. Combination treatments of this nature can be advantageous by alleviating the pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide.

In an embodiment, the compositions disclosed herein further comprise an additional agent capable of alleviating pain in the subject. In an embodiment, the additional analgesic agent is not an agent that binds to LANCL1.

In an embodiment, the additional analgesic agent is capable of alleviating nociceptive pain in the subject. In another embodiment, the additional analgesic agent is capable of alleviating neuropathic pain in the subject.

Suitable agents capable of alleviating nociceptive pain will be familiar to persons skilled in the art, illustrative examples of which are described elsewhere herein and include opiates such as morphine, fentanyl, tramadol, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the additional analgesic agent capable of alleviating nociceptive pain is an opioid. In an embodiment, the additional analgesic agent is an NSAID.

In other embodiments disclosed herein, the compositions disclosed herein are formulated for administration together, either sequentially or in combination (e.g., as an admixture), with another therapy to treat or alleviate pain or the underlying condition that is causing pain. In some instances, the amount of the additional analgesic agent may be reduced when administration is together with the peptides disclosed herein. Illustrative examples of suitable agents capable of treating neuropathic pain are described elsewhere herein.

In other embodiments disclosed herein, the compositions disclosed herein are formulated for administration together, either sequentially or in combination (e.g., as an admixture), with another therapeutic agent for treating any other condition in the subject. In some instances, the amount of the additional therapeutic agent may be reduced when administration is together with the peptides disclosed herein.

Illustrative examples of suitable pharmaceutical formulations include those suitable for enteral or parenteral administration, illustrative examples of which are described elsewhere herein, including oral, rectal, buccal, sublingual, vaginal, nasal, topical (e.g., transdermal), intramuscular, subcutaneous, intravenous, epidural, intra-articular and intrathecal. In an embodiment, the composition is formulated for oral administration.

The therapeutic agents described herein may suitably be placed into the form of pharmaceutical compositions and unit dosages thereof to be employed as solids (e.g., tablets or filled capsules) or liquids (e.g., solutions, suspensions, emulsions, elixirs, or capsules filled with the same) for oral use, in the form of ointments, suppositories or enemas for rectal administration, in the form of sterile injectable solutions for parenteral use (e.g., intramuscular, subcutaneous, intravenous, epidural, intra-articular and intrathecal administration); or in the form of ointments, lotions, creams, gels, patches, sublingual strips or films, and the like for local (e.g., topical, buccal, sublingual, vaginal) administration. In an embodiment, the agents disclosed herein are formulated for topical (e.g., transdermal) delivery. Suitable transdermal delivery systems will be familiar to persons skilled in the art, illustrative examples of which are described by Prausnitz and Langer (2008; Nature Biotechnol. 26(11): 1261-1268), the contents of which are incorporated herein by reference. In another embodiment, the agents disclosed herein are formulated for sublingual or buccal delivery. Suitable sublingual and buccal delivery systems will be familiar to persons skilled in the art, illustrative examples of which include dissolvable strips or films, as described by Bala et al. (2013; Int. J. Pharm. Investig. 3(2):67-76), the contents of which are incorporated herein by reference.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

In some embodiments, it may be desirable to elect a route of administration on the basis of whether the pain or other condition is localized or generalized. For example, where the pain or condition is localized, it may be desirable to formulate the compositions disclosed herein for administration to the affected area or to an area immediately adjacent thereto. For instance, where the pain is in a joint (e.g., neck, knee, elbow, shoulder or hip), the composition can be formulated for intra-articular administration into the affected joint. Alternatively, or in addition, the composition can be formulated for administration at, or substantially adjacent to, the affected joint. As another illustrative example, where the pain is in the oral cavity (e.g., trigeminal neuropathic pain, atypical odontalgia (phantom tooth pain) or burning mouth syndrome), the composition can be formulated for administration via the oral mucosa (e.g., by buccal and/or sublingual administration).

Conversely, where the pain or other condition is generalized or disseminated across multiple anatomical sites of a subject, it may be convenient to formulate the composition for enteral, topical and/or parenteral route of administration, as described elsewhere herein, with a view to distributing the active agents across the multiple anatomical sites affected by pain or condition.

In an embodiment, the composition is formulated for oral administration to a human. In another embodiment, the composition is formulated for oral administration to a non-human subject. In yet another embodiment, the composition is formulated for oral administration to a non-human subject selected from the group consisting of a feline, a canine and an equine.

In another embodiment, the composition is formulated for parenteral administration to a human. In another embodiment, the composition is formulated for parenteral administration to a non-human subject. In yet another embodiment, the composition is formulated for parenteral administration to a non-human subject selected from the group consisting of a feline, a canine and an equine. In an embodiment, the parenteral administration is subcutaneous administration.

In another embodiment, the composition is formulated for topical administration to a human. In another embodiment, the composition is formulated for topical administration to a non-human subject. In yet another embodiment, the composition is formulated for topical administration to a non-human subject selected from the group consisting of a feline, a canine and an equine. In an embodiment, the topical administration is transdermal.

In another embodiment, the composition is formulated as a controlled release dosage form to be administered to a human. In another embodiment, the composition is formulated as a controlled release dosage form to be administered to a non-human subject. In yet another embodiment, the composition is formulated as a controlled release dosage form to be administered to a non-human subject selected from the group consisting of a feline, a canine and an equine. Illustrative examples of suitable controlled release dosage forms are described elsewhere herein.

For preparing the compositions described herein, pharmaceutically acceptable carriers can be either solid or liquid. Illustrative examples of solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier may be a finely divided solid which is in a mixture with the finely divided active component. In tablets, the active component may be mixed with the carrier having the necessary binding capacity in suitable proportions and compacted in the shape and size desired.

In some embodiments, the powders and tablets contain from five or ten to about seventy percent of the active compound. Illustrative examples of suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the actives with encapsulating material, providing a capsule in which the active, with or without carriers, is surrounded by a carrier. Similarly, cachets and lozenges are also envisaged herein. Tablets, powders, capsules, pills, cachets, and lozenges can be used as solid forms suitable for oral administration.

For preparing suppositories, a low melting wax, such as admixture of fatty acid glycerides or cocoa butter, is first melted and the active is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The agents disclosed herein may be suitably formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active compound(s) may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active agent in water and adding suitable colorants, flavours, stabilizing and thickening agents, as desired. Aqueous suspensions suitable for oral use can be made by dispersing the finely divided active agent in water with viscous material, such as natural or synthetic gums, resins, methylcellulose, sodium carboxymethylcellulose, or other well known suspending agents.

Also contemplated herein are solid form preparations which are intended to be converted, shortly before use, to liquid form preparations for oral administration. Such liquid forms include solutions, suspensions, and emulsions. These preparations may contain, in addition to the active agent, colorants, flavours, stabilizers, buffers, artificial and natural sweeteners, dispersants, thickeners, solubilizing agents, and the like.

For topical administration to the epidermis, the therapeutic agents described herein may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising the active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the peptides used in the invention may be encapsulated with cyclodextrins, or formulated with their agents expected to enhance delivery and retention in the nasal mucosa.

Administration to the respiratory tract may also be achieved by means of an aerosol formulation in which the active agent is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, or in addition, the therapeutic agent may be provided in the form of a dry powder, for example a powder mix of the compound in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the agent will generally have a small particle size for example of the order of 1 to 10 microns or less. Such a particle size may be obtained by means known in the art, for example by micronization.

When desired, formulations adapted to give controlled or sustained release of the active agent may be employed, as described elsewhere herein.

In an embodiment, the pharmaceutical preparations, as herein described, are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Also disclosed herein is a composition comprising a therapeutic agent, as herein described, for use as a medicament.

In an embodiment, the compositions disclosed herein are formulated for oral administration to a human. In yet another embodiment, the compositions disclosed herein are formulated for oral administration to a non-human. In a further embodiment, the compositions disclosed herein are formulated for oral administration to a non-human selected from the group consisting of a feline, a canine and an equine.

In another embodiment, the agents disclosed herein are formulated for oral administration to a human subject. In another embodiment, the agents disclosed herein are formulated for oral administration to a non-human subject. In yet another embodiment, the agents disclosed herein are formulated for oral administration to a non-human subject selected from the group consisting of a feline, a canine and an equine.

In another embodiment, the agents disclosed herein are formulated for topical administration to a human subject. In yet another embodiment, the agents disclosed herein are formulated for topical administration to a non-human subject. In another embodiment, the agents disclosed herein are formulated for topical administration to a non-human subject selected from the group consisting of a feline, a canine and an equine. In an embodiment, the topical administration is transdermal.

In another embodiment, the agents disclosed herein are formulated for administration to a human subject as a controlled release dosage form. In yet another embodiment, the agents disclosed herein are formulated for administration to a non-human subject as a controlled release dosage form. In another embodiment, the agents disclosed herein are formulated for administration to a non-human subject as a controlled release dosage form, wherein the non-human subject is selected from the group consisting of a feline, a canine and an equine. In an embodiment, the controlled release dosage form is formulated for parenteral administration.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, the compositions disclosed herein can be suitably formulated for administration via said multiple routes. For example, it may be desirable to administer a first dose parenterally (e.g., intramuscular, intravenously; subcutaneously, etc.) to induce a rapid or otherwise acute analgesic effect in a subject, followed by a subsequent (e.g., second, third, fourth, fifth, etc.) dose administered non-parenterally (e.g., enterally and/or topically) to provide continuing availability of the active agent over an extended period subsequent to the acute phase of treatment. Thus, in an embodiment, the agents and compositions, as disclosed herein, are formulated for parenteral administration to the subject as a first dose (i.e., as a parenteral dosage form) and formulated for non-parenteral administration to the subject after the first dose (e.g., as an enteral and/or topical dosage form). In an embodiment, the parenteral administration is selected from the group consisting of intramuscular, subcutaneous and intravenous. In a further embodiment, the parenteral administration is subcutaneous.

In another embodiment, the enteral administration is oral administration. Thus, in an embodiment, the agents and compositions, as disclosed herein, are formulated for parenteral administration to the subject as a first dose and formulated for oral administration to the subject after the first dose (i.e., as an oral dosage form).

In another embodiment, the enteral administration is topical administration. Thus, in an embodiment, the agents and compositions, as disclosed herein, are formulated for parenteral administration to the subject as a first dose and formulated for topical administration to the subject after the first dose (i.e., as an oral dosage form). In an embodiment, the topical administration is transdermal administration.

In another embodiment, it may be desirable to administer a first dose parenterally (e.g., intramuscular, intravenously; subcutaneously, etc) to induce a rapid or otherwise acute analgesic effect in a subject, followed by a subsequent (e.g., second, third, fourth, fifth, etc.) administration of a controlled release dosage form, as described elsewhere herein, to provide a controlled release of the active agent over an extended period subsequent to the acute phase of treatment. Thus, in another embodiment, the agents and compositions, as disclosed herein, are formulated for parenteral administration to the subject as a first dose and formulated as a controlled release dosage form to be administered to the subject after the first dose. In an embodiment, the controlled release dosage form is formulated for parenteral administration.

It may also be desirable to administer a first dose enterally (e.g., orally or rectally), followed by a subsequent (e.g., second, third, fourth, fifth, etc) dose administered topically (e.g., transdermally). Thus, in an embodiment, the agents and compositions, as disclosed herein, are formulated for enteral administration to the subject as a first dose (i.e., as an enteral dosage form; oral or rectal) and formulated for topical administration to the subject after the first dose (e.g., as a transdermal or transmucosal dosage form). In another embodiment, the agents and compositions, as disclosed herein, are formulated for topical administration selected from the group consisting of transdermal and transmucosal administration. In a further embodiment, the peptides and compositions, as disclosed herein, are formulated for transdermal administration.

In yet another embodiment, it may be desirable to administer the agents or compositions, as disclosed herein, enterally (e.g., orally or rectally) as a first dose, followed by a subsequent (e.g., second, third, fourth, fifth, etc.) dose as a controlled release dosage form, as described elsewhere herein. Thus, in an embodiment, the agents and compositions, as disclosed herein, are formulated for administration as a first dose enterally and formulated for administration as a controlled release dosage form, wherein the controlled release dosage form is formulated for administration subsequent to the first dose. In an embodiment, the enteral dose is formulated for oral administration. In another embodiment, the controlled release dosage form is formulated for parenteral administration.

In an embodiment, it may be desirable to administer the agents or compositions, as disclosed herein, topically (e.g., orally or rectally) as a first dose, followed by a subsequent (e.g., second, third, fourth, fifth, etc.) dose as a controlled release dosage form, as described elsewhere herein. Thus, in an embodiment, the agents and compositions, as disclosed herein, are formulated for topical administration as a first dose and formulated for administration as a controlled release dosage form, wherein the controlled release dosage form is formulated for administration subsequent to the first topical dose. In an embodiment, the topical dose is formulated for transdermal administration. In another embodiment, the controlled release dosage form is formulated for parenteral administration.

Pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragée, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragée, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Pharmaceutical compositions of the present invention for rectal or vaginal administration may be presented as a suppository, which may be prepared by mixing one or more active ingredient(s) with one or more suitable non-irritating carriers which are solid at room temperature, but liquid at body temperature and, therefore, will melt in the rectum or vaginal cavity and release the active compound. Pharmaceutical compositions of the present invention which are suitable for vaginal administration also include pessaries, tampons, creams, gels, pastes, foams or spray formulations containing such pharmaceutically-acceptable carriers as are known in the art to be appropriate.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions of the present invention suitable for parenteral administrations comprise one or more agent(s)/compound(s)/antigen-binding molecules in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

Dosage forms for the topical or transdermal administration include powders, sprays, ointments, pastes, creams, lotions, gels, solutions, patches, drops and inhalants. The active agent (e.g., therapeutic combination) may be mixed under sterile conditions with a suitable pharmaceutically-acceptable carrier. The ointments, pastes, creams and gels may contain excipients. Powders and sprays may contain excipients and propellants.

In some cases, in order to prolong the effect of a pharmaceutical composition, it is desirable to slow its absorption from subcutaneous or intramuscular injection. This may be accomplished by the inclusion of a liquid suspension of crystalline or amorphous material having poor water solubility.

The rate of absorption of individual components of the therapeutic combination then depends upon their rates of dissolution which, in turn, may depend upon crystal size and crystalline form. Alternatively, delayed absorption of a parenterally-administered agent or antibody may be accomplished by dissolving or suspending the active agent or antibody in an oil vehicle. Injectable depot forms may be made by forming microencapsulated matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are given merely to illustrate the present invention and not in any way to limit its scope.

### EXAMPLES

### Example 1: In-Gel Fluorescence to Identify Molecular Targets of Cyclic Peptides

As described elsewhere herein, the present inventors had previously found that peptide fragments of human growth hormone comprising the cyclic region of SEQ ID NO:1, or non-human analogues thereof, are useful for the treatment of pain, including neuropathic pain (see WO2019/136528). More recently, structural analogues of these cyclic peptide fragments have also been shown to have the same or similar properties, including for the treatment of neuropathic pain (see, e.g., WO2019/183686, U.S. patent application No. 62/855,270 and Australian patent application no. 2019902436).

With that information, the present inventors sought to identify and characterise the molecular target for these cyclic peptides.

Using a ligand driven approach, the present inventors utilized the photo-activatable cross-linking PAL from Evotec A.G. (Photo-Activated Labelling) technology that derivatizes the active cyclic peptides to allow UV-induced cross-linking to binding targets (described herein as peptide/SEQ ID NO:X-PAL). This allows subsequent detection in cells, gels of tissue isolates or in pull-down of the peptide/target complex for sequencing by mass spectroscopy (PALMS). The cyclic peptide of SEQ ID NO:12 (LAT9991) was found to be functionally stable when conjugated to the PAL grouping (SEQ ID NO:12-PAL; also referred to herein as LAT9991-PAL), and confirmed to be fully active on the spinal cord slice model (data not shown).

The in-gel fluorescence protocol can be briefly described as follows:

### 1. LANCL1 Labelling, Assay on 96 Well Plate, Volume of the Reaction 40 μl in PBS

3 μg/well of human recombinant LANCL1 (ABCAM catalog no. ab181923, 1 mg/ml; expressed in E. Coli and incorporating an N-terminal 23 amino acid hexaHis tag; UniProtKB/Swiss-Prot Accession no: O43813);

(SEQ ID NO: 56)
vol 3 μl
MGSSHHHHHHSSGLVPRGSHMGSMAQRAFPNPYADYNKSLAEGYFD
AAGRLTPEFSQRLTNKIRELLQQMERGLKSADPRDGTGYTGWAGIA
VLYLHLYDVFGDPAYLQLAHGYVKQSLNCLTKRSITFLCGDAGPLA
VAAVLYHKMNNEKQAEDCITRLIHLNKIDPHAPNEMLYGRIGYIYA
LLFVNKNFGVEKIPQSHIQQICETILTSGENLARKRNFTAKSPLMY
EWYQEYYVGAAHGLAGIYYYLMQPSLQVSQGKLHSLVKPSVDYVCQ
LKFPSGNYPPCIGDNRDLLVHWCHGAPGVIYMLIQAYKVFREEKYL
CDAYQCADVIWQYGLLKKGYGLCHGSAGNAYAFLTLYNLTQDMKYL
YRACKFAEWCLEYGEHGCRTPDTPFSLFEGMAGTIYFLADLLVPTK
ARFPAFEL,

Vehicle DMSO
±LAT8881 (a cyclic peptide fragment of human growth hormone; SEQ ID NO:1)
SEQ ID NO:1) as competitor @ 25, 50, 100 or 200 μM, pre-incubation 10 min, 4 μl of a stock solution 10-fold concentrated
±LAT9991-PAL, LAT7771-PAL or LAT9993-PAL probes @ 1 μM, incubation 30 min at room temperature on a plate agitator (protected from light), 4 μl of a stock solution @10 μM

### 2. LANCL1 Photolabelling

Irradiation @ 365 nm 20 min (4° C., on a support containing ice) Transfer 40 μl in a clean Eppendorf tube

### 3. Reduction & Alkylation

Reduction: DTT 10 mM, 30 min @ 56° C.
Alkylation: Iodoacetamide 30 mM, 45 min @ RT Click-iT AzideTamra of 60 μL samples

### 4. Click-Reaction

Acetone Precipitation −20° C. ON
Dry pellet resuspended in in 30 μL 1% SDS in 50 mM Tris-HCl pH 7.5
Photolabeled LANCL1 was tagged with tetramethylrhodamine (TAMRA) azide @100 μM TAMRA azide by copper click chemistry using the Click-iT Protein Reaction Buffer Kit (ThermoFisher Scientific) during 30 minutes
Precipitation using the chloroform-methanol method, pellet was air-dried for 10 min at room temperature, resuspended in 30 μL SDS loading buffer (Bio Rad's XT Sample Buffer containing 2.5% v/v 2-mercaptoethanol) and heated (60° C., 30 min)

### 5. Gel-Based Analysis of Cross-Linked Proteins

SDS-PAGE (4-15% Criterion™ TGX Stain-Free™ Protein Gel, Bio Rad)
Analyzed by in-gel fluorescence scanning using a ChemiDoc™ MP Imaging System (Bio Rad) with a green LED light as an excitation source and a BP600/20 nm emission filter

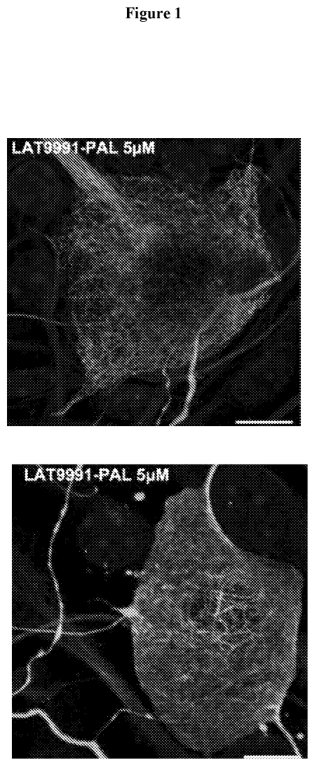

The cell-binding fluorescence protocol can be briefly described as follows. Binding of LAT9991-PAL can be seen in DRG neurons from mice treated with paclitaxel (neuropathic pain model), but not in control untreated mice (FIG. 1). Model of chemotherapy-induced peripheral neuropathy was generated using paclitaxel in female C57/B16 mice—50 mg/kg Paclitaxel or vehicle (10 ml/kg body weight) injected intravenously (i.v.) on day 1, day 3 and day 5. Mechanical allodynia was confirmed using von Frey (VF) filaments 9 days after starting vehicle or paclitaxel treatment (left and right paws). Each of the ten C57/B16 mice (vehicle control and paclitaxel treated) were euthanized by CO₂ inhalation and individually dissected.

L5-L6 DRGs were rapidly dissected and placed in Petri dishes containing dissection medium. Meninges were removed.

Each DRG pool was successively:
incubated in dissociation buffer (0.5 mg/ml Dispase, 2.5 mg/ml collagenase, 6 mg/ml BSA, 10 mM HEPES) for 30 min at 37° C. under gentle agitation at 800 rounds/min;
washed;
triturated in culture medium and filtered on a 40 μm filter;
re-suspended in 1 ml culture medium supplemented with nerve growth factor (NGF) 250 ng/ml.

Dissociated cells from each pool were counted, seeded on Poly-D-Lysin- and Laminin-coated μ slides at the density of 4×10⁴ cells/well and incubated at 37° C. 5% CO₂. The medium was changed daily for 3 days. After 3 days of culture, DRG cells from each condition (Control or Paclitaxel) were incubated for an hour, at 37° C., 5% CO₂ with the following treatment:
PBS (Control)
LAT9991-PAL at 2.5 μM
LAT9991-PAL at 2.5 μM+competitor LAT8881 at 50 μM
LAT9991-PAL at 2.5 μM+competitor LAT9991 at 50 μM
LAT9991-PAL at 5 μM
LAT9991-PAL at 5+competitor LAT8881 at 50 μM
LAT9991-PAL at 5+competitor LAT9991 at 50 Mm

Culture medium was removed and replaced by PBS. Cells were UV light irradiated (365 (365 nm) for 20 min on ice to cross-link LAT9991-PAL to target proteins. Cells were fixed with Paraformaldehyde 3.7% 15 min at RT (Mol. Probes R37602). Localization of binding of the LAT-9991-PAL ligand was revealed by immunofluorescence under confocal microscopy.

LAT9991-PAL was used in conjunction with confocal microscopy to visualise the location of potential target(s), noting that analgesic agents can have activity at several sites, either on central or peripheral neurons, or on glial or inflammatory cells that are in proximity to neurons. To identify the cell types to which LAT9991-PAL binds, 3-day cell cultures of DRG from neuropathic or control sites were taken from animals with neuropathy due to spinal nerve ligation. Briefly, L5 and L6 spinal nerves of male Sprague Dawley rats were ligated to create a robust and long-lasting mechanical allodynia of the injured hind paw. Presence of neuropathic pain was confirmed 2 weeks post-spinal nerve ligation surgeries (day 14) using von Frey (VF) filaments (up-and-down method) applied on ipsilateral (injured) and contralateral (non-injured) paw). Live DRG cells were dissected out as described previously and incubated with LAT9991-PAL for 1 hour prior to UV irradiation to fix the binding in situ. Over several repeated experiments, it was shown that LAT9991-PAL localised only in neurons from neuropathic DRG and not from unaffected DRG (either the contralateral DRG in a constriction model or DRG from healthy control animals in chemotherapy models). As a specificity control, the binding of LAT9991-PAL to the neuropathic DRG was blocked in the presence of an excess amount of unlabeled LAT9991 peptide.

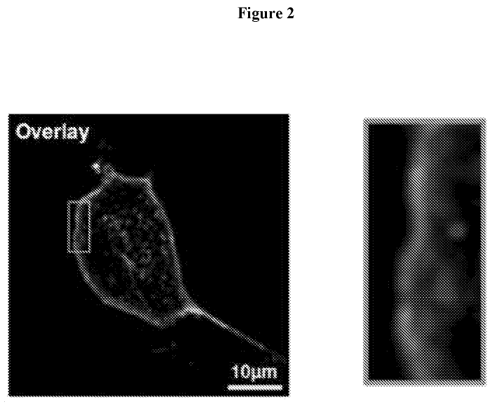

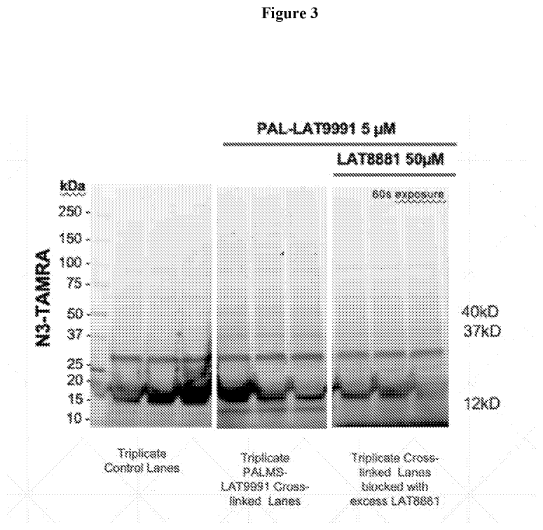

High-powered imaging showed that the target for LAT9991-PAL was expressed just inside the neuronal cell membrane, and with punctate staining in the cytoplasm (FIG. 2).

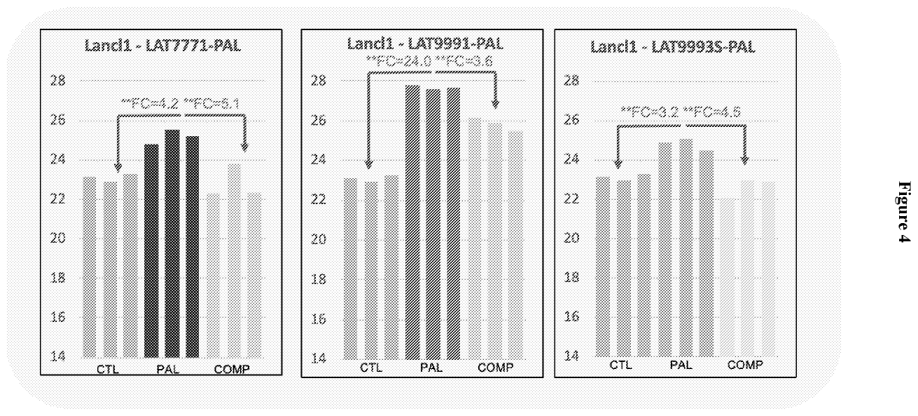

LAT9991-PAL was cross-linked to tissue homogenates of nerve from neuropathic animals. After separation on gels, specific patterns of staining were revealed that identify targets at 3 molecular weight ranges: ˜12-15 kD, ˜37 kD and ˜50 kD (FIG. 3). Of note, cyclic peptide conjugates LAT9993-PAL and LAT7771-PAL were also shown to cross-link to tissue homogenates of nerve from neuropathic animals.

Having identified at least three molecular weight bands as representative of the potential targets, the 12-15 kD, 37 kD and 50 kD regions of the gel were excised, subjected to limited proteolysis and analysed by mass spectrometry to identify peptides from proteins that were differentially enriched. The detection of at least two different peptides from a protein that is enriched in the LAT9991-PAL cross-linked samples when compared to (i) the un-crosslinked samples and (ii) LAT9991-PAL in the presence of excess LAT8881 (SEQ ID NO:1; YLRIVQCRSVEGSCGF) was considered a positive signature. LAT9991-PAL, LAT9993-PAL and LAT7771-PAL were also used. LAT9993 (SEQ ID NO:41) has the amino acid sequence SCRSRPVESSC.

From the 37 kDa band, analysis by mass spectrometry identified LANCL1 as a candidate and met the arbitrary statistical criteria for a “hit” (for binding to LAT9991-PAL), as described above (the LANCL1-derived peptides, underlined and bold below, were identified):

MAQR [symbol] [symbol] LTNKIR
ELLQQMERGLKSADPQDGTGYTGWAGIAVLYLHLHNVFGDPAY
LQMAHSYVK [symbol] R [symbol] [symbol]
MNSGK [symbol] LIHLNKIDPHVPNEMLYGRIGYIFALLF
VNK [symbol] LSRKRNFTTKSPL
MYEWYQEYYVGAAHGLAGIYYYLMOPSLHVSQGKLHS [symbol] [symbol] [symbol]
DLLVHWCHGAPGVIYMLIQAYK
VFKEEHYLCDAQQCADVIWQYGLLKKGYGLCHGAAGNAYAFL
ALYNLTODAKYLYRACK [symbol] HGCRTPDTPFSLF
EGMAGTIYFLADLLVPTKAKFPAFEL).

Note that LANCL1 was also detected in the LAT9993-PAL and LAT7771-PAL samples. FIG. 4 shows that LANCL1 was enriched by several fold-changes in the LAT7771-PAL, LAT9991-PAL and LAT9993-PAL samples over the control un-crosslinked samples (CU) and samples incubated with excess LAT8881 (COMP) (the Y axis is provided for log base 2 scale).

To confirm the LANCL1 enrichment observed by mass spectrometry sequencing pull-down experiments, commercial antibodies to LANCL1 (polyclonal rabbit anti-LANCL1, PA557107 from Invitrogen) were used to identify the presence of the LANCL1 in nerve microsome preparations. Briefly, rat spinal cord were lysed in lysate buffer, (sodium phosphate buffer 5 mM pH7.4, sucrose 0.32M+protease inhibitors (cOmplete ULTRA tablets from Roche, mini easy pack, 05892970001)). All the following steps were done on ice. The samples were homogenized, and the total extract centrifuged at 10000 g during 20 minutes. The supernatant was kept and the lysis step was repeated on the pellet adding 10 ml more of lysis buffer, followed by a new centrifugation step. This step extraction was done two times as described and the supernatants were pooled before centrifugation at 105000 g using the Ti50.2 Beckman rotor for 90 minutes at 4° C. The pellet was kept as a microsomal fraction and stored at −80° C. The concentration of protein of the microsomal fraction was 8.8 mg/ml. the microsomal fraction was treated with three different conditions, by incubation with one of the following: 1) DMSO control; 2) 5 μM LAT9991-PAL probe; 3) pre-treatment with 50 μM LAT8881, as competitor 10-fold in excess and LAT9991-PAL probe; followed by UV-irradiation to initiate photo-crosslinking (20 minutes at 365 nm). Subsequently, probe-labeled proteins were subjected to the click reaction through the aliphatic alkyne functional group on the probe with biotin azide probe so that the probe-labeled protein(s) were selectively tagged with a biotin reporter (according to manufacturer's instructions; Thermo Fischer Click-iT Assay kits). An input sample was removed and reserved. The remaining reaction volume was added to strepavidin magnetic bead slurry reactions to purify the biotin-labelled proteins. Flow through fraction was reserved, and the beads washed twice with 50 mM Tris, 150 mM NaCl pH 7.5, 2 M urea followed by two washes with 50 mM ammonium bicarbonate. The beads were then re-suspended in 50 mM ammonium bicarbonate and used in Western Blot analyses, using rabbit anti-LANCL1 (Invitrogen PA-57107 antibody) and goat anti-rabbit (E Bioscience ref-18881633). The blots were subsequently probed with an HRP conjugate and visualised by enhanced chemiluminescence (SuperSignal West Dura substrate, ThermoFisher) and documented with the ChemiDoc™ MP Imaging System (Bio Rad). Images acquired with the ChemiDoc™ MP Imaging System were analyzed with Imagelab software (Bio-Rad).

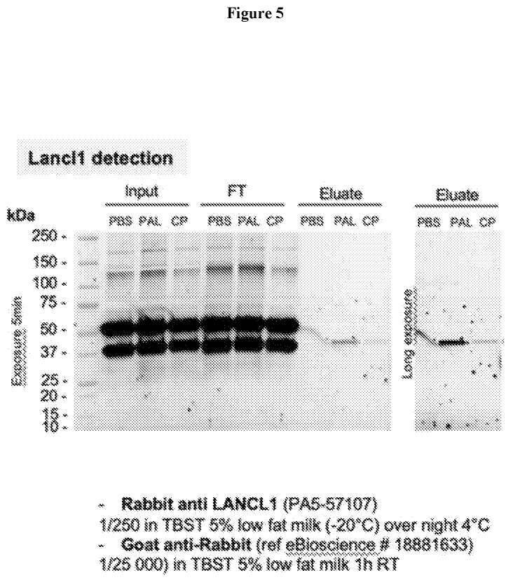

LANCL1 was detected in the input, flow through (FT) of the negative control PBS samples, the LAT9991-PAL sample and the LAT9991-PAL sample that had been incubated with excess LAT8881 (see FIG. 5). These data confirm that LANCL1 is expressed in nerve cells. However, LANCL1 could only be detected in the eluates of LAT9991-PAL samples as a specifically bound protein and levels were reduced in the presence of excess LAT8881 (see CP lane in FIG. 5).

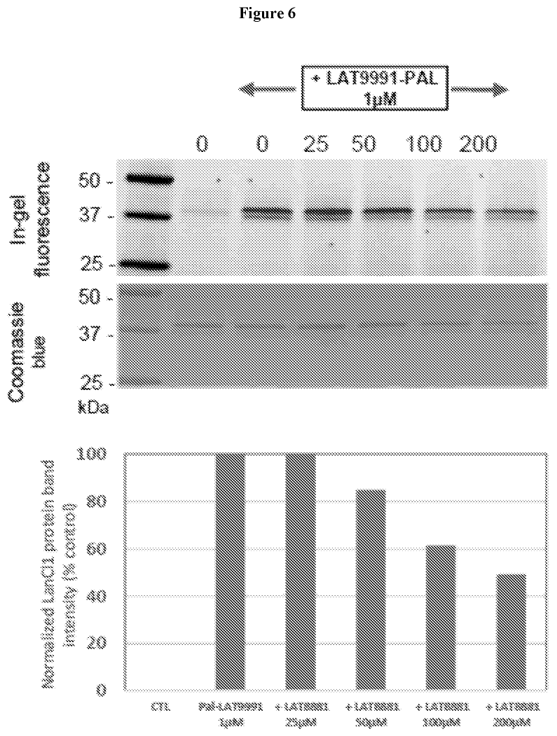

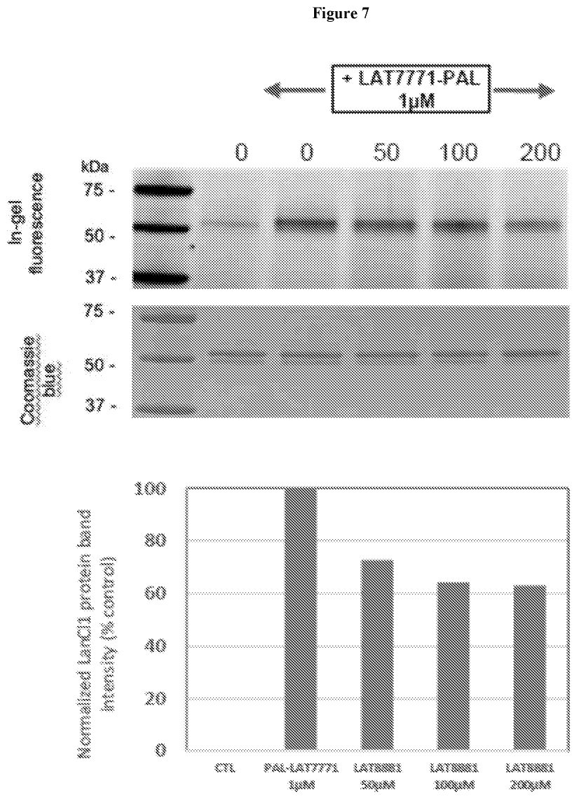

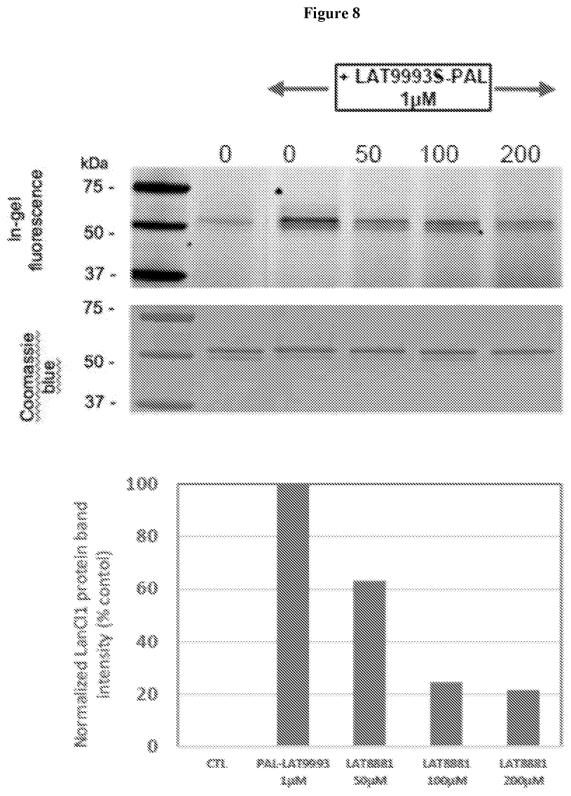

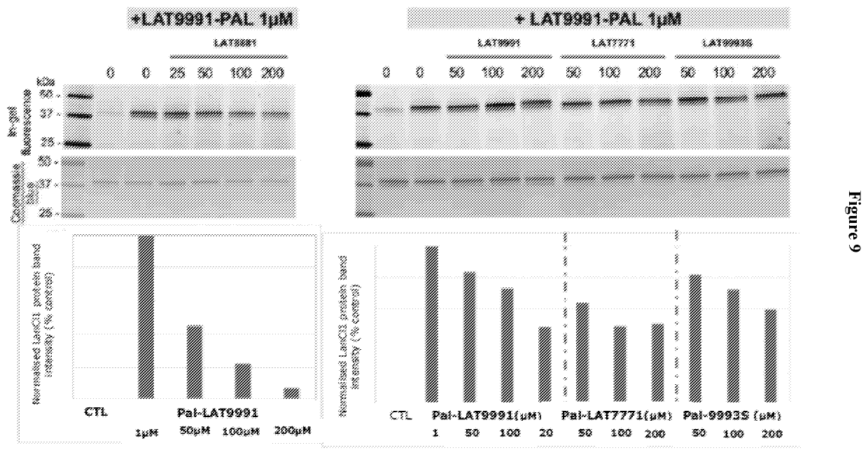

In a further study, LAT9991-PAL, LAT7771-PAL and LAT9993S-PAL were cross-linked to tissue homogenates of nerve from neuropathic animals. After separation on gels, specific patterns of staining again revealed the targets at 3 molecular weight ranges: ˜12-15 kD, ˜37 kD and ˜50 kD. LAT9991-PAL, LAT7771-PAL and LAT9993S-PAL were each shown to cross-link to tissue homogenates of nerve from neuropathic animals. The binding of cyclic peptides LAT9991-PAL, LAT7771-PAL and LAT9993S-PAL to recombinant LANCL1 was inhibited in the presence of excess LAT8881 (see FIGS. 6-8, respectively).

In a parallel study, LAT9991-PAL was cross-linked to tissue homogenates of nerve from neuropathic animals, as evidenced by the specific patterns of staining showing 3 molecular weight ranges: ˜12-15 kD, ˜37 kD and ˜50 kD.

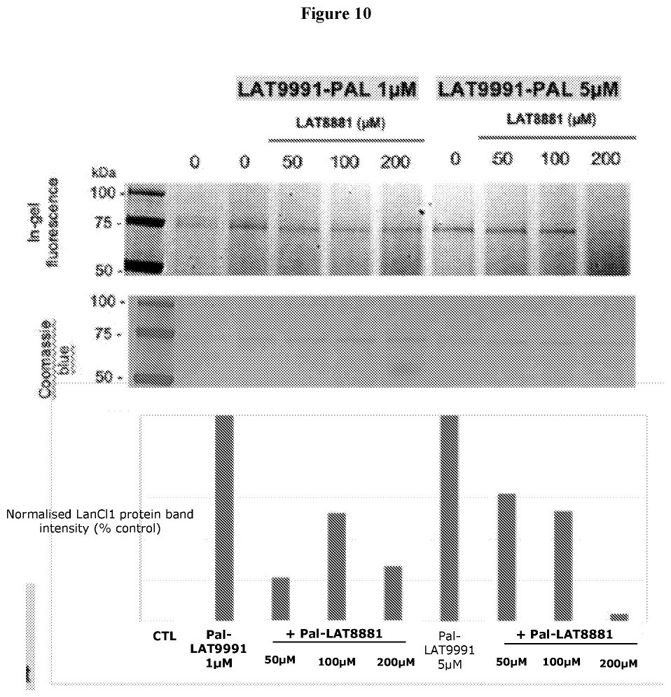

The binding of LAT9991-PAL to recombinant LANCL1 was inhibited in the presence of excess LAT8881, LAT9991, LAT7771 and LAT9993S (see FIG. 9).

### Example 2: In-Gel Fluorescence to Identify Binding of Cyclic Peptides to LANCL2 and LANCL3

An in-gel fluorescence protocol was followed to determine whether the cyclic peptides bound to LANCL2 and LANCL3. Briefly:

### 1. LANCL2 and LANCL3 Labelling, Assay on 96 Well Plate, Volume of the Reaction 40 μl in PBS

1 μg/well of human recombinant LANCL2 (amino acid residues 1-450; ABCAM catalog no. ab163277, ˜0.07 mg/ml; expressed in wheat germ and incorporating an N-terminal GST tag; UniProt Accession no: Q9NS86);

SEQ ID NO: 57)
vol 3 μl
MGETMSKRLKLHLGGEAEMEERAFVNPFPDYEAAAGALLASGAAEE
TGCVRPPATTDEPGLPFHQDGKIIHNFIRRIQTKIKDLLQQMEEGL
KTADPHDCSAYTGWTGIALLYLQLYRVTCDQTYLLRSLDYVKRTLR
NLNGRRVTFLCGDAGPLAVGAVIYHKLRSDCESQECVTKLLQLQRS
VVCQESDLPDELLYGRAGYLYALLYLNTEIGPGTVCESAIKEVVNA
IIESGKTLSREERKTERCPLLYQWHRKQYVGAAHGMAGIYYMLMQP
AAKVDQETLTEMVKPSIDYVRHKKFRSGNYPSSLSNETDRLVHWCH
GAPGVIHMLMQAYKVFKEEKYLKEAMECSDVIWQRGLLRKGYGICH
GTAGNGYSFLSLYRLTQDKKYLYRACKFAEWCLDYGAHGCRIPDRP
YSLFEGMAGAIHFLSDVLGPETSRFPAFELDSSKRD;,

3 μg/well of human recombinant LANCL3 (amino acid residues 1-420; ABCAM catalog no. ab163277, ˜0.07 mg/ml; expressed in E. Coli and incorporating an N-terminal 10×His tag and a C-terminal Mvc tag; UniProt Accession no: Q6ZV70);

SEQ ID NO: 58)
vol 3 μl
MDTKRCFANRFDDYQGSLLAGQCEEAVAPLVTATIERILQELPPL
GGGAEARGATAGASACQGGLYGGVAGVAYMLYHVSQSPLFATARE
RYLRSAKRLIDACARAEEWGEPDADTRAAFLLGGAGVYAVATLVY
HALGRSDYVQPLGKFRALCAVCAPVSFLECGSDELFVGRAGYLCA
ALVLKQKLAQEVLTPAQIKSICQAILDSGKQYAIKKRKPFPLMYS
YYGTEYLGAAHGLSSILQMLLSYHEHLKPSDRELVWQSVDFLMEQ
EQNCNWPPELGETIERENELVHWCHGAPGIAYLFAKAYLVSKKPQ
YLDTCIRCGELTWQKGLLKKGPGICHGVAGSAYVFLLLYRLTGNS
KYIYRAQRFAQFLFTEEFKAGSRVLESIYSLYEGFSGTVCFLIDL
LQPNQAEFPLFSVFV;,

Vehicle DMSO
±LAT8881 (a cyclic peptide fragment of human growth hormone; SEQ ID NO:1)
SEQ ID NO:1 as competitor @ 50, 100 or 200 μM, pre-incubation 10 min, 4 μl of a stock solution 10-fold concentrated
±LAT9991-PAL probes @ 1 μM, incubation 30 min at room temperature on a plate agitator (protected from light), 4 μl of a stock solution @10 μM

### 2. LANCL2 and LANCL3 Photolabelling

Irradiation @ 365 nm 20 min (4° C., on a support containing ice)
Transfer 40 μl in a clean Eppendorf tube

### 3. Reduction & Alkylation

Reduction: DTT 10 mM, 30 min @ 56° C.
Alkylation: Iodoacétamide 30 mM, 45 min @ RT Click-iT AzideTamra of 60 μL samples

### 4. Click-Reaction

Acetone Precipitation −20° C. ON
Dry pellet resuspended in in 30 μL 1% SDS in 50 mM Tris-HCl pH 7.5
Photolabeled LANCL1 was tagged with tetramethylrhodamine (TAMRA) azide @100 μM TAMRA azide by copper click chemistry using the Click-iT Protein Reaction Buffer Kit (ThermoFisher Scientific) for 30 minutes
Precipitation using the chloroform-methanol method, pellet was air-dried for 10 min at room temperature, resuspended in 30 μL SDS loading buffer (Bio Rad XT Sample Buffer containing 2.5% v/v 2-mercaptoethanol) and heated (60° C., 30 min)

### 5. Gel-Based Analysis of Cross-Linked Proteins

SDS-PAGE (4-15% Criterion™ TGX Stain-Free™ Protein Gel, Bio Rad)
Analyzed by in-gel fluorescence scanning using a ChemiDoc™ MP Imaging System (Bio Rad) with a green LED light as an excitation source and a BP600/20 nm emission filter

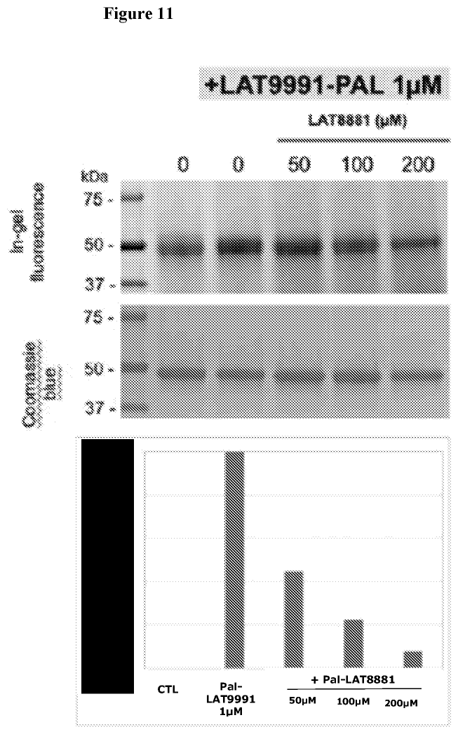

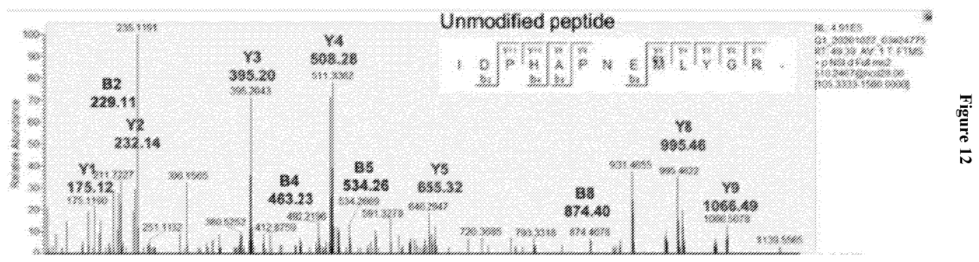

As shown in FIGS. 10 and 11, LAT9991-PAL binds to LANCL2 and LANCL3 and that said binding is inhibited by the presence of excess LAT8881.

### Example 3: In-Vitro Photolabelling of 6His-LanCL1 with LAT9991-PAL Probe and LC-MS/MS Analysis to Identify a Specific Binding Site

### A. Preparation of Labelled LANCL1 for MS-Analysis

Recombinant human 6His-LaNCL1 protein (2 μM, 1 nmol, 53 μg) in 530 μL PBS was pre-incubated with 50 μM of LAT8881 or DMSO for 10 min and then treated with 25 μM LAT9991-PAL for another 30 min at RT in Nunc™ MicroWell™ 96-well plates (Thermo Fisher Scientific cat #167008) (final reaction volume, 530 μL). The samples were UV-irradiated (365 nm) for 20 min at 4° C. After UV-irradiation, the samples were split in two different samples containing 3 μg or 50 μg of protein.

### B. Control of Protein Photolabelling by in Gel Fluorescence Scanning

Protein samples (3 μg protein) were adjusted to 1% SDS and 10 mM DTT. After incubation for 1 h at 56° C., protein samples were treated with 30 mM iodoacetamide for 45 min at RT in the dark. Dry acetone (9 volumes) prechilled to −20° C. was added and the cloudy mixture was vortexed thoroughly and incubated at −20° C. overnight. After centrifugation (15,000×g for 10 min at 4° C.), the supernatants were poured off and the remaining pellets washed with −20° C. acetone. The wash supernatants were removed by centrifugation and the precipitated protein pellets were air-dried for 10 min at RT and resuspended in 30 μL of 1% SDS in 50 mM Tris-HCl, pH 7.5. Probe-labelled LANCL1 was tagged with 100 μM tetramethylrhodamine (TAMRA) azide (Thermo Fisher Scientific cat #T10182) by copper click chemistry using the #according to the manufacturer's instructions. Proteins were then precipitated using the chloroform-methanol method described by Wessel and Flügge (Wessel and Flügge, 1984) and the precipitated protein pellets were air-dried for 10 min at room temperature. After removing the supernatants, the protein pellets were air-dried and dissolved in laemmli buffer, heated at 60° C. for 20 minutes and analyzed by in gel fluorescence scanning

### C. Labelled LANCL1 for MS-Analysis

The remaining 50 μg of protein samples were precipitated by Dry acetone (9 volumes) prechilled to −20° C., the cloudy mixture was vortexed thoroughly and incubated at −20° C. overnight. After centrifugation, the pellets were solubilized in 30 μL of 6M Urea in 50 mM NH4HCO3 and sonicated 3 times for 10 seconds. The samples were reduced by with 10 mM DTT during 60 minutes at room temperature and alkylated with 30 mM iodoacetamide or 45 min at RT in the dark. Samples were diluted with 50 mM NH4HCO3 pH 8.0 and then digested by a final enzyme:substrate ratio of 1:25 (w/w), this was incubated at 37° C. overnight under mild agitation. Peptide mixtures (50 μg) were further acidified (1% TFA final concentration), and cleaned-up using Bond Elut OMIX Pipette-based SPE C18 tips (Agilent cat #A57003100). First, pipette tips were pre-conditioned using 100 μL of 50% ACN, and equilibrated with 100 μl of 0.1% TFA in H20. The peptide mixtures were loaded by dispending and aspirating the samples 10 times and washed twice with 100 μL of 0.1% TFA in H20. Elution was performed successively with 100 μl of 50% ACN/0.1% TFA and 100 μl of 80% ACN/0.1% TFA. The eluates were then pooled and evaporated under vacuum. Before LC-MS/MS analysis, peptides were resuspended in 10 μL of 0.2% FA/5% DMSO. The resulting peptides were subsequently sonicated in a water bath for 15 min and mixed at RT for 5 min.

### D. LC-MS/MS Analysis of LANCL1 Peptides

Peptides were analyzed by nanoLC-MS/MS with the Ultimate 3000 RSLC (Thermo Fisher Scientific) coupled online to a Q-Exactive Plus mass spectrometer with a NanoFlex source. Analytical columns (40 cm long, 75 μm ID) were packed in-house with ReproSil-Pur 120 C18-AQ, 1.9 μm reversed phase resin (Dr Maisch GmbH cat #r119.aq) with emitters pulled using a P-2000 Laser Based Micropipette Puller System (Sutter Instrument). To reduce the backpressure at high flow rates and to enhance separation efficiency, the column compartment was kept at 60° C. The peptide mixture (5 μl) was loaded onto the analytical column with 5% solvent B (80% ACN, 5% DMSO, 0.2% FA) in solvent A (5% DMSO, 0.2% FA) at a flow rate of 400 nL/min and separated with a linear gradient of 5% to 30% solvent B at a flow rate of 300 nL/min over 103 min. Due to the loading, lead-in, and washing steps, the total time for an LC-MS/MS run was about 180 min. The Q-Exactive Plus was operated in data-dependent acquisition mode using the following settings: full-scan automatic gain control (AGC) target 3×106 at 70,000 resolution; scan range 350-1500 m/z; Orbitrap full-scan maximum injection time 45 ms; MS2 scan AGC target 3.2×103 at 17,500 resolution; maximum injection 45 ms; normalized collision energy 27; dynamic exclusion time 30 s; isolation window 2.2 m/z; 10 MS2 scans per full scan.

### E. MS Data Processing

The raw files were processed with the MaxQuant software for peptide and protein identification and quantification. MS/MS raw files of the digests were searched using the Andromeda search engine against a database containing only the recombinant human 6HisLANCL1 sequence using the following parameters: carbamidomethylation of cysteine was set as fixed modification whereas N-terminal acetylation and methionine oxidation were set as variable modifications. All peptides were required to have a minimum peptide length of seven amino acids and a maximum of two miss cleavages. Specificity for Glu-C cleavage was required allowing cleavage after glutamate and aspartate. The mass tolerances were set to 4.5 ppm and 20 ppm in MS and MS/MS, respectively. The false discovery rate (FDR) for protein and peptide identifications was set to a maximum of 1%. To validate and transfer identifications across different runs, the “match between runs” option in MaxQuant was enabled with a Match time window of 0.7 min and an alignment time window of 20 min. Unknown modifications were identified by the “dependent peptides” setting implemented in MaxQuant in a standard search (Cox et al., 2011; J. Am. Soc. Mass Spectrom. 22:1373-1380). The algorithm performs an unbiased search for modified peptides that are derived from an already identified peptide. If an unidentified spectrum matches an identified spectrum, the mass shift (corresponding to the modification of the peptide) between the theoretical and the observed precursor mass and the matched sequence will be reported. Modified peptides will be only identified if they are derived from an already identified unmodified peptide with a FDR of 1% and a mass tolerance of 6.5 mDa. Modified peptides were extracted from allPeptides.txt along with the ΔM mass shift between the unmodified “base peptide” and the modified peptide. All amino acids were considered as possible residues for modification. The mass of the modification used to search for probe-modified peptides was +454.1991 m/z for LAT9991-PAL which is the mass for the corresponding probe minus two nitrogen atoms and cleaved by the trypsin/Lys C enzymes after the arginine amino acid. This modification was set as a variable modification in all MaxQuant searches. In brief, for “dependent peptides” analysis, the “all.peptides.txt” file was loaded and filtered for DP Proteins=“sp|DLanCL1|”, DP Mass Difference=454.1991+/−6 ppm and DP Score “>60”. Selected peptides with a DP mass shift corresponding to the photoadduct with a tolerance of 6 ppm and which are only present in the two conditions “LAT9991-PAL” and “LAT9991-PAL+LAT8881” and absent in the control “DMSO” were considered as positive hits. Remaining hits were further validated in a manual fashion. MS spectra were visualized with the Xcalibur software to validate the presence of the unmodified and modified peptides. Ideally, the unmodified peptide should be detected in all three conditions whereas the peptide modified with a photo-adduct should be detected in the condition “LAT9991-PAL” and to a lesser extent in the condition “LAT9991-PAL+LAT8881” but not in the control “DMS0”. MS2 spectra were visualized using the viewer program of MaxQuant to annotate y and b ions of the unmodified peptide. MS2 spectra of the unmodified and modified peptides of interest were analyzed using XCalibur to determine the position of the photo-adduct in the sequence. A mass shift corresponding to the photo-adduct on a y and/or a b ion is expected.

### Results:

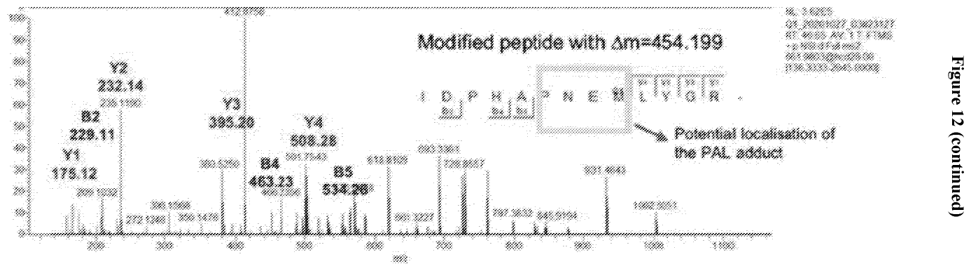

The only modified peptide sequence differentially detected in the LAT9991-PAL condition was IDPHAPNEM(ox)LYGR, with the PNEM sequence (amino acid residues 171-174 of human LANCL1 (SEQ ID NO:56) the most likely site for localization of the LAT9991-PAL adduct (see FIG. 12).

### Example 4: Co-Localization of LANCL1 and LAT9991F-PAL Binding on Respiratory Epithelial Cells

### A. Cell Imaging

A photoactivatable analog of LAT8881 (LAT9991F (SEQ ID NO:13)-PAL), was used to conduct the experiments. A549 or NCI-H358 cells were treated with LAT9991F-PAL at 2.5 μM during 30 minutes. Fluorescent signal was recorded after that LAT9991F-PAL was immobilized on its target(s) site by UV-crosslinking and the LAT9991F-PAL-target complexes were labeled with the green-fluorescent Alexa Fluor 488 alkyne dye by click-chemistry. Control cells were treated with the vehicle, dimethyl sulfoxide (DMSO), and with LAT9991F-PAL in combination with a large excess of the parent drug LAT8881 or other competitors, GSH, NSC61610 or ABA to evaluate, respectively, the non-specific fluorescent background and the specificity of the staining. For colocalization experiments, subsequently, immunofluorescence staining with an anti-LANCL1 or LANCL2 antibodies and an Alexa 568 (red) conjugated secondary antibody was performed. Images stacks of cells labeled with probes were collected and evaluated for the degree of colocalization of LAT9991F-PAL-target complexes and LANCL1 or LANCL2 using either the Measure Colocalization plugin in Metamorph or the JACoP plugin in ImageJ).

TABLE 3
LIST OF ABBREVIATIONS

| | |
|---|---|
| DMEM | Dulbecco’s modified eagle medium |
| DMSO | Dimethyl sulfoxide |
| FBS | Foetal bovine serum |
| LanCL1 | Glutathione S-transferase LANCL1 |
| LanCL2 | Glutathione S-transferase LANCL2 |
| NSC | NSC61610 |
| GSH | L-Glutathione reduced protein |
| ABA | Abscisic acid |
| PAL | Photoaffinity labeling |
| PBS | Phosphate buffer solution |
| PCC | Pearson's correlation coefficient |
| ROI | Region-of-interest |
| UV | Ultraviolet |

### Materials and Methods

### 1. Cell Line and Culture Conditions

A549 or NCI-H358 cells (ATCC CCL-185 and ATCC CRL-5807, respectively) were cultured in appropriate medium containing 10% heat-inactivated FBS (Dutscher, #SV30160-036), 1% penicillin/streptomycin (Gibco, #15140-122) and maintained in a humidified 37° C. incubator with 5% CO₂.

TABLE 4
CELL CULTURE MEDIA

| Cell line | Culture medium |
|---|---|
| A549 | DMEM GIBCO réf: 119600 500 ml |
| ATCC | Pyruvate de Sodium GIBCO 100 mM réf: 11360-039_5 ml, 1 mM final |
| CCL-185 | GlutaMax GIBCO 35050-087 5 ml 1 mM final |
| | MEM NEAA 1% |
| | Pennicilin/Streptomycin 100 × Gibco concentrations finales: 100 |
| | IU/ml penicillin, 100 μg/ml streptomycin |
| | SVF 10% Gibco réf: 10270 50 ml |
| NCI-H358 | DMEM GIBCO réf: 119600 500 ml |
| ATCC | Pyruvate de Sodium GIBCO 100 mM réf: 11360-039_5 ml, 1 mM final |
| CRL-5807 | GlutaMax GIBCO 35050-087 5 ml 1 mM final |
| | Pennicilin/Streptomycin 100 × Gibco concentrations finales: 100 |
| | IU/ml penicillin, 100 μg/ml streptomycin |
| | SVF10% Gibco réf: 10270 50 ml |

### 2. Tested Items

LAT9991F-PAL is a photosenzitive compound, all the manipulations of LAT9991F-PAL were carried out in the dark as far as possible. bLAT8881 was provided by Lateral PHARMA (Melbourne, Australia). LAT9991F-PAL was synthesized at Evotec, Toulouse.

TABLE 5
CHARACTERISTICS OF TESTED ITEMS

| | | Stock | |
|---|---|---|---|
| | Molecular Weight | concentration | |
| Corporate name | (g/mol) | (mM) | Solvent |
| LAT8881 | 1813.86 | 10 | DMSO |
| LAT9991F-PAL | 1042.3 | 10 | DMSO |
| Taxol (Sigma) T7402 | 853.91 | 10 | DMSO |
| H₂O₂ (Sigma) H1009 | 34.01 | 10 | DMSO |
| GSH (Sigma) G6013 | 307.32 | 10 | DMSO |
| NSC61610 (Sigma) | 548.59 | 10 | DMSO |
| SML2293 | | | |
| Abscisic acid | 264.32 | 10 | DMSO |
| (Sigma) SLCD2431 | | | |

### 3. Equipment

Centrifuge 1-15 pk (Sigma)
Direct heat CO₂ incubator (Thermo Electron)
UVP CL-1000 UV crosslinking chamber (Hyland Scientific)
Axiovert 200M microscope (Zeiss) with a CSU-W1 Yokogawa confocal unit

### 4. Tested Items Preparation

LAT9991F-PAL was dissolved in DMSO to make a 10 mM stock solution. Intermediate solutions at 25 μM and 2.5 μM were made in the culture medium from the 10 mM stock solutions of LAT8881 and LAT9991F-PAL, respectively. A two-fold dilution into PBS of each intermediate solution was used for cell treatment. A stock solution at 10 mM was prepared for the other competitors as GSH, NSC61610 and ABA. A final concentration at 25 μM was used for the assay.

### 5. Histochemical Staining

ibidiTreat Polymer Coverslips (Ibidi, #80826) were coated with serum during 1 hour at 37° C., followed by Laminin (100 μg\ml) (incubation for 1 hour at 37° C.) for NCI-H358 cells.

A549 or NCI-H358 cells were seeded onto ibidiTreat Polymer Coverslips (Ibidi, #80826) at 2.5×10⁵ cells/cm² in 200 μl of complete medium. After 24 hours, the medium was removed and cells were washed with PBS (Gibco, #10010049) and then pre-treated with the Taxol at 1 μM for 3 hours or with H₂O₂ 25 μM for 2 hours. Cells were washed twice with PBS. Induced stress cells or control cells were then exposed to LAT9991F-PAL (2.5 μM) in PBS for 30 minutes, in the presence or absence of LAT8881 (25 μM), or the other compounds used as a competitor, and pre-incubated 10 minutes. After treatment, cells were washed with cold PBS and UV-irradiated (365 nm) in PBS for 20 min. After fixation with 4% paraformaldehyde and permeabilization (0.5% Triton/PBS) (Image-iT™ Fixation/Permeabilization Kit, ThermoFisher, #R37602), the bioorthogonal reaction with Alexa Fluor 488 Alkyne (1 μM) (ThermoFisher, #A10267) was performed at 25° C. using the Click-iT™ Cell Reaction Buffer Kit (ThermoFisher, #C10269) according to the manufacturer protocol. After click reaction, cells were washed twice with PBS, blocked with 3% BSA in PBS for 1 hour and stained with Hoechst 33342 dye (ThermoFisher, #H1399).

For colocalization experiments, cells were further incubated overnight at 4° C. with a mouse anti-LanCL1 antibody (Invitrogen, PAS-57-107) (dilution 1:1000) or LanCL2 (Atlas Antibodies HPA019711) (dilution 1:1000) in 3% BSA in PBS, washed twice with PBS, and then incubated respectively with a goat anti-mouse Alexa 488 (Molecular Probes, #A21467) (dilution 1:800) for 1 h in 3% BSA in PBS. After three further washing steps with PBS, cells were embedded in Ibidi Mounting Medium (ibidi, #50001). The specimens were kept at 4° C. in the dark until analysis.

### 6. Microscopy and Image Analysis

Images were acquired on an Axiovert200M (Zeiss) microscope using a CSU-W1 Yokogawa confocal unit. This microscope is equipped with a plan Apochromat X40 dry objective numerical aperture (NA) 0.95 and a Neofluar X100 oil immersion objective NA 1.45. Images were capture with an EMCCD camera (Prior, ProEM 1024×1024). To account for different experimental setups (i.e., quantification of LAT9991F-PAL-target complexes, LANCL1), laser powers and exposure settings were adjusted individually and kept constant throughout the whole measurement allowing comparability. Images were acquired with Metamorph software (Molecular Devices). Integrated intensity (II) and surface (A) were measured for each fluorescent dye in z-stack images. Briefly, background fluorescence was subtracted at all slices of the z-stack. Then, for each channel, a threshold defined for DMSO-treated cell images was applied to all LAT9991F-PAL-treated cell images analyzed in order to quantify the internalized fluorescent signal. A region-of-interest (ROI) was defined for each object and II as well as A were measured using Metamorph software. The II/A ratio was determined for each condition. A one-way ANOVA associated with a Dunnett's multiple comparisons test (GraphPad Prism Version 7.0) was used to compare LAT9991F-PAL-treatment or LAT9991F-PAL+LAT8881-treatment and LAT9991F-PAL+other competitor treatments with DMSO for each condition, with or without induced stress. For colocalization analysis, z-stack images were analyzed either with the Measure Colocalization plugin in Metamorph software to determine the percentage of overlap between two objects across all pixels in the ROI in an image or with the JACoP plugin in ImageJ software (Bolte and Cordelieres 2006) to calculate the Pearson's correlation coefficient (PCC). PCC is measured across all pixels in the ROI in an image. PCC values range from −1 to +1. If there is no colocalization of the two probes, the expected PCC is 0. A positive PCC means that the two probes colocalize to some extent. For advanced 3D visualization, images were processed with Avizo Fire 3D visualization and analysis software (FEI).

7. LANCL Knockdown by siRNA

ibidiTreat Polymer Coverslips (Ibidi, #80826) were coated with serum during 1 hour at 37° C., followed by Laminin (100 μg\ml) (incubation 1h at 37° C.) for NCI-H358 cells. A549 or NCI-H358 cells were seeded onto ibidiTreat Polymer Coverslips (Ibidi, #80826) at 2.5×10⁵ cells/cm² in complete medium, in a final volume of 200 μl. A549 or NCI-H358 cells were transfected using the kit Lipofectamine RNAi-MAX Transfection Reagent (Thermo Fisher LMRNA015). After 24 h, the media was removed, cells were washed twice with PBS, and complete medium was added. The mix solution for the transfection was prepared, 5 ul of siRNA control (SiControl) or siLanCL1 (Silencing of Glutathione 5-transferase LANCL1) stock solution at 20 μM) was added at 125 μl of OptiMEM medium, (final concentration at 100 nM) as solution A. si-RNA was from Dharmacon: ON-TARGET plus human LANCL1 (10314) siRNA—SMART POOL (L-012166-00-0005).

A solution of lipofectamine RNAI max reagent, 3 μl was added at 125 μl OptiMEM medium, as solution B. Solution A and solution B were mixed, vortexed and incubated during 20 minutes. The mixing solution, 200 μl was added on the cells. After 24 hours, the medium was removed, washed twice with PBS and complete medium with serum was added. After 24 hours more, the medium was removed and the treatments were done.

### Results

### 1. A549 Adenocarcinomic Alveolar Basal Epithelial Cell Line

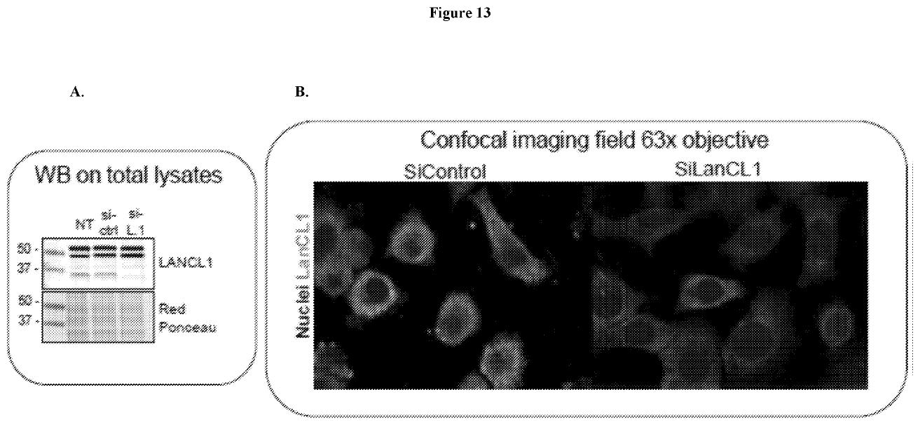

Confocal microscopy showed co-localization of LANCL1 and LAT9991F-PAL binding in the adenocarcinomic alveolar basal epithelial cell line, A549. Co-localization of LANCL1 and LAT9991F-PAL was lost following siRNA knockdown of LANCL1 in A549 cells (SiLANCL1 A549 cells). This was despite incomplete silencing of LANCL1 via siRNA, as evidenced by weak detection of endogenous LANCL1 in the cytosol of the SiLANCL1 A549 cells (see FIG. 13).

LAT9991F-PAL binding was detected in siLANCL1 A549 cells following induced oxidative stress with either hydrogen peroxide (H202; 25 μM for 2 hours) or Taxol (1 μM for 3 hours), but the level of binding was less intense as compared to control cells (SiControl).

LAT9991F-PAL binding on SiLANCL1 A549 cells was partially competed off in the presence of excess LAT8881 (25 μM), LAT9993S (25 μM), LAT7771 (25 μM) and glutathione (GSH; 25 μM) when compared to control cells. By contrast, LAT9991F-PAL binding on SiLANCL1 A549 cells was strongly competed off by the LANCL2 ligand, NSC61610, in all conditions tested.

### 2. NCI-H358 Non-Small Cell Lung Cancer Cell Line

Confocal microscopy showed co-localization of LANCL1 and LAT9991F-PAL binding to the non-small cell lung cancer cell line, NCI-H358. Co-localization of LANCL1 and LAT9991F-PAL was lost following siRNA knockdown of LANCL1 in the NCI-H358 cells (SiLANCL1 NCI-H358 cells). This was despite incomplete silencing of LANCL1 via siRNA, as evidenced by weak detection of endogenous LANCL1 in the cytosol of the SiLANCL1 NCI-H358 cells.

LAT9991F-PAL binding was detected in siLANCL1 NCI-H358 cells following stress, but the level of binding was less intense as compared to control cells.

LAT9991F-PAL binding on SiLANCL1 NCI-H358 cells was partially competed off in the presence of excess LAT8881 (25 μM), LAT9993S (25 μM), LAT7771 (25 μM) and glutathione (GSH; 25 μM) when compared to control cells. By contrast, LAT9991F-PAL binding on SiLANCL1 NCI-H358 cells was strongly competed off by NSC61610 in all conditions tested.

These data confirm that LANCL1 is a putative target of the cyclic peptides disclosed herein, including LAT8881, LAT9991, LAT9991F, LAT9993 and LAT7771.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A method of treating pain in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising
SEQ ID NO: 1
(YLRIVQCRSVEGSCGF)

2. The method of claim 1, wherein the pain is neuropathic pain.

3. The method of claim 2, wherein the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, migraine, drug-induced peripheral neuropathy and peripheral neuropathy associated with infection, chronic low back pain, complex regional pain syndrome, temporomandibular joint disorders, Lichen Planus and reflex sympathetic dystrophy.

4. The method of any one of claims 1 to 3, further comprising administering to the subject in need thereof an additional analgesic agent, wherein the additional analgesic agent is not (i) an agent that binds to LANCL1, or (ii) an agent that competes for binding to LANCL1 with a cyclic peptide comprising SEQ ID NO: 1.

5. The method of claim 4, wherein the additional analgesic agent comprises an agent capable of alleviating nociceptive pain in a subject.

6. The method of claim 5, wherein the additional analgesic agent is an opioid.

7. The method of claim 5, wherein the additional analgesic agent is selected from the group consisting of morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID).

8. The method of claim 7, wherein the NSAID is selected from the group consisting of aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelonean.

9. The method of any one of claims 1 to 8, wherein the agent is not a peptide derived from human interleukin-1 receptor-associated kinase 3 (IRAK-3).

10. The method of any one of claims 1 to 8, wherein the agent is not a peptide derived from human prolactin.

11. A method of screening for analgesic agents, the method comprising: (a) contacting a candidate agent with a Lanthionine synthetase C-like protein (LANCL) in the presence of a cyclic peptide comprising SEQ ID NO:1, or a structural analogue thereof, and under conditions that would allow binding of the candidate agent to the LANCL, and (b) determining whether the candidate agent binds to the LANCL and competes for binding to the LANCL with the cyclic peptide comprising SEQ ID NO:1 or with the structural analogue thereof, wherein the ability of the candidate agent to compete for binding to the LANCL with the cyclic peptide comprising SEQ ID NO:1, or with the structural analogue thereof, is indicative that the candidate agent is an analgesic agent.

12. A method of claim 11, wherein the structural analogue of the cyclic peptide comprising SEQ ID NO:1 is derived from human interleukin-1 receptor-associated kinase 3 (IRAK-3).

13. A method of claim 11, wherein the structural analogue of the cyclic peptide comprising SEQ ID NO:1 is derived from human prolactin.

14. The method of claim 11, wherein the structural analogue comprises the peptide of formula (I):
R¹-CRSVEGSCG-R² (I)
wherein
R¹ is selected from the group consisting of YLRIVQ, LRIVQ, RIVQ, IVQ, VQ, and Q, or R¹ is absent; and
R² is F (phenylalanine), or R² is absent,
wherein the peptide of formula (I) is a cyclic peptide formed by a disulphide bond between the two cysteine residues.

15. The method of claim 14, wherein the structural analogue is selected from the group consisting of LRIVQCRSVEGSCGF (SEQ ID NO:11), CRSVEGSCG (SEQ ID NO:12), CRSVEGSCGF (SEQ ID NO:13) and a cyclic peptide comprising an amino acid sequence having at least 70% sequence identity to any of the foregoing.

16. The method of claim 11, wherein the structural analogue comprises the peptide of formula (II):
R¹-C-R-X¹-X²-P-X³-X⁴-X⁵-X⁶-C-R² (II)
wherein
X¹, X³, X⁵, and X⁶ is an amino acid residue selected from the group consisting of serine, alanine, valine, leucine, isoleucine and glycine;
X² is alanine, arginine or lysine;
X⁴ is glutamic acid or aspartic acid;
R¹ is selected from the group consisting of:
S,
HS,
GHS,
PGHS,
APGHS,
EAPGHS,
SEAPGHS,
SSEAPGHS,
PSSEAPGHS,
DPSSEAPGHS,
and
IDPSSEAPGHS,
or R¹ is absent; and
R² is selected from the group consisting of:
S,
ss,
SSK,
SSKF,
SSKFS,
SSKFSW,
SSKFSWD,
SSKFSWDE,
SSKFSWDEY,
SSKFSWDEYE,
SSKFSWDEYEQ,
SSKFSWDEYEQY,
SSKFSWDEYEQYK,
SSKFSWDEYEQYKK,
and
SSKFSWDEYEQYKKE,
or R² is absent; and
wherein the peptide of formula (II) is a cyclic peptide formed by a disulphide bond between the two cysteine residues.

17. The method of claim 16, wherein the structural analogue is selected from the group consisting of YLRVMKCRRFVESSCAF, LRVMKCRRFVESSCAF, CRRFVESSCAF, CRRFVESSCA and a cyclic peptide comprising an amino acid sequence having at least 70% sequence identity to any of the foregoing.

18. The method of claim 11, wherein the structural analogue comprises the peptide of formula (III):
R¹-C-R-I-X₁-X₂-X₃-X₄-N-C-R² (III)
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent,
wherein the peptide of formula (III) is a cyclic peptide formed by a disulphide bond between the two cysteine residues.

19. The method of claim 18, wherein the structural analogue is selected from the group consisting of CRIIHNNNC, CRIIHNNNCG, CRIVYDSNC, CRIVYDSNCG and a cyclic peptide comprising an amino acid sequence having at least 70% sequence identity to any of the foregoing.

20. The method of any one of claims 11 to 19, further comprising isolating, synthesizing or otherwise producing the candidate agent identified as an analgesic agent.

21. The method of any one of claims 11 to 20, wherein the LANCL is selected from the group consisting of LANCL1, LANCL2 and LANCL3.

22. The method of claim 21, wherein the LANCL is LANCL1.

23. A composition comprising the analgesic agent identified by the method according to any one of claims 11 to 22, wherein the agent is not a peptide derived from human growth hormone or from a non-human homologue thereof.

24. The composition of claim 23, further comprising a pharmaceutically acceptable carrier.

25. The composition of claim 23 or claim 24, wherein the agent is not a peptide derived from human interleukin-1 receptor-associated kinase 3 (IRAK-3).

26. The composition of claim 23 or claim 24, wherein the agent is not a peptide derived from human prolactin.

27. The composition of any one of claims 23 to 26, further comprising an additional analgesic agent, wherein the analgesic agent is not an agent that binds to LANCL1.

28. The composition of claim 27, wherein the additional analgesic agent is an agent capable of alleviating nociceptive pain in a subject.

29. The composition of claim 28, wherein the additional analgesic agent is opioid.

30. The composition of claim 28, wherein the additional analgesic agent is selected from the group consisting of morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID).

31. A composition for use in treating pain in a subject in need thereof, the composition comprising an agent that binds to Lanthionine synthetase C-like protein 1 (LANCL1), and competes for binding to LANCL1 with a cyclic peptide comprising SEQ ID NO:1 (YLRIVQCRSVEGSCGF), or with a structural analogue thereof, and wherein the agent is not a peptide derived from human growth hormone or from a non-human homologue thereof.

32. The composition for use of claim 31, wherein the pain is neuropathic pain.

33. The composition for use of claim 32, wherein the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, migraine, drug-induced peripheral neuropathy and peripheral neuropathy associated with infection, chronic low back pain, complex regional pain syndrome, temporomandibular joint disorders, Lichen Planus and reflex sympathetic dystrophy.

34. The composition for use of any one of claims 31 to 33, further comprising an additional analgesic agent, wherein the additional analgesic agent is not (i) an agent that binds to LANCL1, or (ii) an agent that competes for binding to LANCL1, or to a homologue thereof, with a cyclic peptide comprising SEQ ID NO:1.

35. The composition for use of claim 34, wherein the additional analgesic agent comprises an agent capable of alleviating nociceptive pain in a subject.

36. The composition for use of claim 35, wherein the additional analgesic agent is an opioid.

37. The composition for use of claim 35, wherein the additional analgesic agent is selected from the group consisting of morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID).

38. The composition for use of any one of claims 31 to 37, wherein the agent is not a peptide derived from human interleukin-1 receptor-associated kinase 3 (IRAK-3).

39. The composition for use of any one of claims 31 to 37, wherein the agent is not a peptide derived from human prolactin.

40. Use of an agent that binds to Lanthionine synthetase C-like protein 1 (LANCL1) and competes for binding to LANCL1 with a cyclic peptide of SEQ ID NO:1 (YLRIVQCRSVEGSCGF), or with a structural analogue thereof, in the manufacture of a medicament for treating pain in a subject in need thereof, wherein the agent is not a peptide derived from human growth hormone or from a non-human homologue thereof and wherein the agent.

41. The use of claim 40, wherein the pain is neuropathic pain.

42. The use of claim 41, wherein the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, migraine, drug-induced peripheral neuropathy and peripheral neuropathy associated with infection, chronic low back pain, complex regional pain syndrome, temporomandibular joint disorders, Lichen Planus and reflex sympathetic dystrophy.

43. The use of any one of claims 40 to 42, wherein the agent is not a peptide derived from human interleukin-1 receptor-associated kinase 3 (IRAK-3).

44. The use of any one of claims 40 to 42, wherein the agent is not a peptide derived from human prolactin.

45. The use of any one of claims 40 to 44, wherein the medicament is formulated for administration with an additional analgesic agent, wherein the additional analgesic agent is not (i) an agent that binds to LANCL1, or (ii) an agent that competes for binding to LANCL1 with a cyclic peptide comprising SEQ ID NO:1.

46. The use of claim 45, wherein the additional analgesic agent comprises an agent capable of alleviating nociceptive pain in a subject.

47. The use of claim 46, wherein the additional analgesic agent is an opioid.

48. The use of claim 46, wherein the additional analgesic agent is selected from the group consisting of morphine, fentanyl, tramadol, codeine, dihydrocodeine, hydrocodone, acetyldihydrocodeine, oxycodone, oxymorphone and buprenorphine, and a non-steroidal anti-inflammatory drug (NSAID).

49. A method of screening for a ligand of a Lanthionine synthetase C-like protein (LANCL), the method comprising: (a) contacting a candidate agent with the LANCL in the presence of a cyclic peptide comprising SEQ ID NO:1, or a structural analogue thereof, and under conditions that would allow binding of the candidate agent to the LANCL, and (b) determining whether the candidate agent binds to the LANCL and competes for binding to the LANCL with the cyclic peptide comprising SEQ ID NO:1 or with the structural analogue thereof, wherein the ability of the candidate agent to compete for binding to the LANCL with the cyclic peptide comprising SEQ ID NO:1, or with the structural analogue thereof, is indicative that the candidate agent is a ligand of LANCL.

50. A method of claim 49, wherein the structural analogue of the cyclic peptide comprising SEQ ID NO:1 is derived from human interleukin-1 receptor-associated kinase 3 (IRAK-3).

51. A method of claim 49, wherein the structural analogue of the cyclic peptide comprising SEQ ID NO:1 is derived from human prolactin.

52. The method of claim 49 wherein the structural analogue comprises the peptide of formula (I):
R¹-CRSVEGSCG-R² (I)
wherein
R¹ is selected from the group consisting of YLRIVQ, LRIVQ, RIVQ, IVQ, VQ, and Q, or R¹ is absent; and
R² is F (phenylalanine), or R² is absent,
wherein the peptide of formula (I) is a cyclic peptide formed by a disulphide bond between the two cysteine residues.

53. The method of claim 14, wherein the structural analogue is selected from the group consisting of LRIVQCRSVEGSCGF (SEQ ID NO:11), CRSVEGSCG (SEQ ID NO:12), CRSVEGSCGF (SEQ ID NO:13) and a cyclic peptide comprising an amino acid sequence having at least 70% sequence identity to any of the foregoing.

54. The method of claim 49, wherein the structural analogue comprises the peptide of formula (II):
R¹-C-R-X¹-X²-P-X³-X⁴-X⁵-X⁶-C-R² (II)
wherein
X¹, X³, X⁵, and X⁶ is an amino acid residue selected from the group consisting of serine, alanine, valine, leucine, isoleucine and glycine;
X² is alanine, arginine or lysine;
X⁴ is glutamic acid or aspartic acid;
R¹ is selected from the group consisting of:
S,
HS,
GHS,
PGHS,
APGHS,
EAPGHS,
SEAPGHS,
SSEAPGHS,
PS SEAPGHS,
DPSSEAPGHS,
and
IDPSSEAPGHS,
or R¹ is absent; and
R² is selected from the group consisting of:
S,
ss,
SSK,
SSKF,
SSKFS,
SSKFSW,
SSKFSWD,
SSKFSWDE,
SSKFSWD EY,
SSKFSWDEYE,
SSKFSWDEYEQ,
SSKFSWDEYEQY,
SSKFSWDEYEQYK,
SSKFSWDEYEQYKK,
and
SSKFSWDEYEQYKKE,
or R² is absent; and
wherein the peptide of formula (II) is a cyclic peptide formed by a disulphide bond between the two cysteine residues.

55. The method of claim 54, wherein the structural analogue is selected from the group consisting of YLRVMKCRRFVESSCAF, LRVMKCRRFVESSCAF, CRRFVESSCAF, CRRFVESSCA and a cyclic peptide comprising an amino acid sequence having at least 70% sequence identity to any of the foregoing.

56. The method of claim 49, wherein the structural analogue comprises the peptide of formula (III):
R¹-C-R-I-X₁-X₂-X₃-X₄-N-C-R² (III)
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent,
wherein the peptide of formula (III) is a cyclic peptide formed by a disulphide bond between the two cysteine residues.

57. The method of claim 56, wherein the structural analogue is selected from the group consisting of CRIIHNNNC, CRIIHNNNCG, CRIVYDSNC, CRIVYDSNCG and a cyclic peptide comprising an amino acid sequence having at least 70% sequence identity to any of the foregoing.

58. The method of any one of claims 49 to 57, further comprising isolating, synthesizing or otherwise producing the candidate agent identified as a ligand of LANCL.

59. The method of any one of claims 49 to 58, wherein the LANCL is selected from the group consisting of LANCL1, LANCL2 and LANCL3.

60. The method of claim 59, wherein the LANCL is LANCL1.

61. A composition comprising the ligand identified by the method according to any one of claims 49 to 60, wherein the ligand is not a peptide derived from human growth hormone or from a non-human homologue thereof.

62. The composition of claim 60 or claim 61, wherein the ligand is not a peptide derived from human interleukin-1 receptor-associated kinase 3 (IRAK-3).

63. The composition of claim 23 or claim 24, wherein the ligand is not a peptide derived from human prolactin.

64. A method of treating a condition in a subject, the method comprising administering to a subject in need thereof a therapeutically effective amount of an agent that binds to Lanthionine synthetase C-like protein 1 (LANCL1), wherein the agent is not a peptide derived from human growth hormone, or from a non-human homologue thereof, and wherein the agent competes for binding to LANCL1 with a cyclic peptide comprising SEQ ID NO:1 (YLRIVQCRSVEGSCGF), wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, a respiratory condition and a bone disorder.

65. The method of claim 64, wherein the agent is not a peptide derived from an interleukin-1 receptor-associated kinase 3 (IRAK-3).

66. The method of claim 64 or claim 65, wherein the agent is not a peptide derived from human prolactin.

67. The method of any one of claims 64 to 66, wherein the condition is a respiratory condition.

68. The method of claim 67, wherein the respiratory condition is selected from the group consisting of chronic obstructive pulmonary disease, asthma, cystic fibrosis and lung cancer and a respiratory tract infection.

69. The method of claim 68, wherein the respiratory condition is a respiratory tract infection.

70. The method of claim 69, wherein the respiratory tract infection is a virus infection.

71. The method of claim 70, wherein the virus is selected from the group consisting of a picornavirus, a coronavirus, an influenza virus, a parainfluenza virus, a respiratory syncytial virus, an adenovirus, an enterovirus, and a metapneumovirus.

72. The method of claim 71, wherein the virus is an influenza virus or a coronavirus.
